(19) United States
(12) Patent Application Publication
(10) Pub. No.: US 2025/0166148 A1
(43) Pub. Date: May 22, 2025

(54) LEARNING DEVICE, PREDICTION DEVICE, LEARNING METHOD, PROGRAM, AND LEARNING SYSTEM

(71) Applicant: J-OIL MILLS, Inc., Tokyo (JP)

(72) Inventors: Kenichi KAKIMOTO, Tokyo (JP); Ryohei WATANABE, Tokyo (JP); Ayato TAKASAKI, Tokyo (JP); Masami INOUE, Tokyo (JP)

(21) Appl. No.: 18/272,609

(22) PCT Filed: Jan. 18, 2022
(86) PCT No.: PCT/JP2022/001589

§ 371 Date: Jan. 24, 2025

(30) Foreign Application Priority Data
Feb. 1, 2021 (JP) JP20210014398

(51) Int. Cl. G06T 7/00; G06N 20/00; G06V 10/70

(52) U.S. Cl. CPC G06T 7/0002; G06N 20/00; G06V 10/70; G06T 2207/20081; G06T 2207/30242

(57) ABSTRACT

According to the present invention, AI for predicting the rate of deterioration is generated. A learning device comprises: an imaging section configured to acquire an image in which edible oil is captured; a state identification section configured to analyze the image to identify a state of the edible oil; and a learning section configured to cause a learning model to learn a correlation between the state and a rate of deterioration indicating an increased level of deterioration of the edible oil.

### TECHNICAL FIELD

The present invention relates to a learning device, a prediction device, a learning method, a program, and a learning system.

### BACKGROUND ART

Appropriately managing the quality of edible oil in cooking of deep-fried foods (hereinafter, referred to as “deep-fry cooking”) preferably enables the quality of deep-fried foods to be kept. Conventionally, it has been known that the more the edible oil is used, the more it deteriorates. For objectively determining the time for changing the edible oil, a method of determining an increased level of deterioration of the edible oil (hereinafter, referred to as “rate of deterioration”, also may be referred to as “level of deterioration”) by referring to the appearance, odor, color tone, variation in these of the edible oil, or a cumulative time (also referred to as “cumulative period of time”) of use of the frying oil has been available.

However, this conventional technique often depends on, for example, the experience (hereinafter, referred to as “subjectivity”) of a person who is in charge of determination (in many cases, such a person is a user of the edible oil). In this respect, as a method for objectively determining the rate of deterioration of the edible oil without depending on the subjectivity, for example, Patent Literature 1 discloses a method of detecting the rate of deterioration by using, as an indicator, variation in the illuminance of the surface of edible oil during deep-fry cooking.

### CITATION LIST

### Patent Literature

Patent Literature 1: JP-A-H08-182624

### SUMMARY OF INVENTION

### Technical Problem

The method according to Patent Literature 1 is employed, for example, in stores and shops, such as convenience stores or supermarkets, which sell, to customers, deep-fried foods produced by deep-fry cooking using cooking tools equipped therein. However, the environment in which such cooking tools are installed differs from store to store, and thus the degree of change in illumination and other factors also vary from store to store. This makes it difficult to accurately predict the rate of deterioration using the method according to Patent Literature 1.

Therefore, an object of the present invention is to provide a learning device for generating Artificial Intelligence (hereinafter, referred to as “AI”) capable of predicting the rate of deterioration.

### Solution to Problem

In order to achieve the object described above, provided is a learning device comprising: an imaging section configured to acquire an image in which edible oil is captured; a state identification section configured to analyze the image to identify a state of the edible oil; and a learning section configured to cause a learning model to learn a correlation between the state and a rate of deterioration indicating an increased level of deterioration of the edible oil.

### Advantageous Effects of Invention

According to the present invention, it is possible to generate AI for predicting the rate of deterioration. The problems, configurations, and advantageous effects other than those described above will be clarified by explanation of the embodiments below.

### BRIEF DESCRIPTION OF DRAWINGS

FIG. 1 illustrates an example of arrangement in a cooking area 1.

FIG. 2 illustrates an example of a hardware configuration of an information processing device.

FIG. 3 illustrates an example of entire processing.

FIG. 4 illustrates examples of bubbles in new edible oil.

FIG. 5 illustrates examples of bubbles in old edible oil.

FIG. 6 illustrates the results of experiment of “intensity of foaming” in the first period.

FIG. 7 illustrates a correlation between “intensity of foaming” and acid values in the first period.

FIG. 8 illustrates the results of experiment of “quantity of form” in the first period.

FIG. 9 illustrates a correlation between “quantity of form” and acid values in the first period.

FIG. 10 illustrates the results of experiment of “size of foam” in the first period.

FIG. 11 illustrates a correlation between “size of foam” and acid values in the first period.

FIG. 12 illustrates the results of experiment of “speed at which foam disappears” in the first period.

FIG. 13 illustrates a correlation between “speed at which foam disappears” and acid values in the first period.

FIG. 14 illustrates the results of experiment of “intensity of foaming” in the second period.

FIG. 15 illustrates a correlation between “intensity of foaming” and acid values in the second period.

FIG. 16 illustrates the results of experiment of “quantity of form” in the second period.

FIG. 17 illustrates a correlation between “quantity of form” and acid values in the second period.

FIG. 18 illustrates the results of experiment of “size of foam” in the second period.

FIG. 19 illustrates a correlation between “size of foam” and acid values in the second period.

FIG. 20 illustrates the results of experiment of “speed at which foam disappears” in the second period.

FIG. 21 illustrates a correlation between “speed at which foam disappears” and acid values in the second period.

FIG. 22 illustrates the results of experiment of “intensity of foaming” in the third period.

FIG. 23 illustrates a correlation between “intensity of foaming” and acid values in the third period.

FIG. 24 illustrates the results of experiment of “quantity of form” in the third period.

FIG. 25 illustrates a correlation between “quantity of form” and acid values in the third period.

FIG. 26 illustrates the results of experiment of “size of foam” in the third period.

FIG. 27 illustrates a correlation between “size of foam” and acid values in the third period.

FIG. 28 illustrates the results of experiment of “speed at which foam disappears” in the third period.

FIG. 29 illustrates a correlation between “speed at which foam disappears” and acid values in the third period.

FIG. 30 illustrates the results of experiment of “intensity of foaming” in the fourth period.

FIG. 31 illustrates a correlation between “intensity of foaming” and acid values in the fourth period.

FIG. 32 illustrates the results of experiment of “quantity of form” in the fourth period.

FIG. 33 illustrates a correlation between “quantity of form” and acid values in the fourth period.

FIG. 34 illustrates the results of experiment of “size of foam” in the fourth period.

FIG. 35 illustrates a correlation between “size of foam” and acid values in the fourth period.

FIG. 36 illustrates the results of experiment of “speed at which foam disappears” in the fourth period.

FIG. 37 illustrates a correlation between “speed at which foam disappears” and acid values in the fourth period.

FIG. 38 illustrates an example of a functional configuration of a learning device.

FIG. 39 illustrates an example of use of a learning system 7.

FIG. 40 illustrates an example of a network architecture.

FIG. 41 illustrates fried foods which are subjects of experiments.

FIG. 42 illustrates the results of experiments obtained by measurement of acid values after 10 hours.

FIG. 43 illustrates the results of experiments using color.

FIG. 44 illustrates the results of experiments using acid values.

### DESCRIPTION OF EMBODIMENTS

Hereinafter, an object to be cooked by deep-fry cooking using edible oil is referred to as “fried food”. The fried foods are, for example, fried chickens, croquettes, French fries, tempuras, pork cutlets, or the like.

### Example of Arrangement in Cooking Area 1

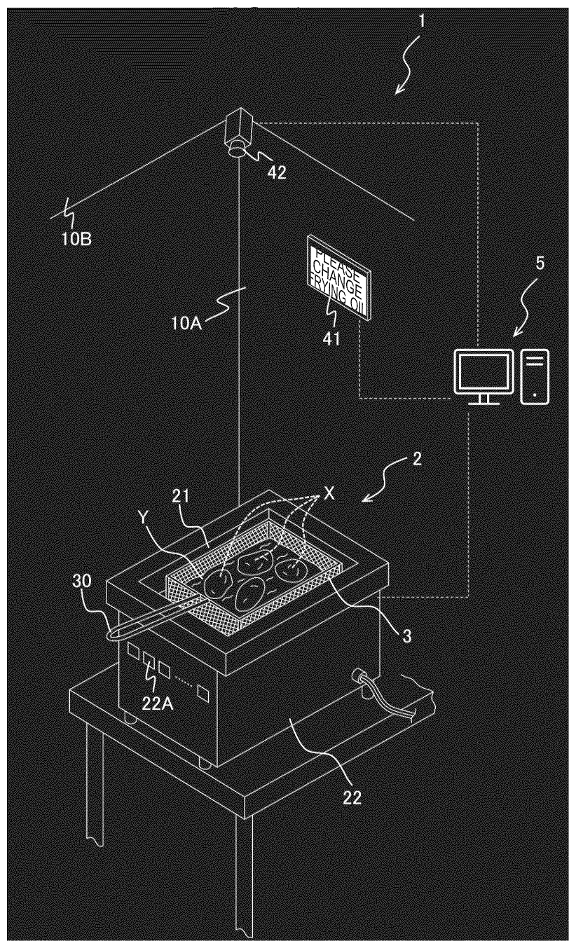

Firstly, an example of arrangement in a cooking area 1, which is assumed as an environment of deep-fry cooking, will be described with reference to FIG. 1.

FIG. 1 illustrates an example of arrangement in the cooking area 1. In the following, edible oil is referred to as “frying oil”.

The cooking area 1 is built within a store such as a convenience store or a supermarket. The cooking area 1 is provided with cooking facilities for deep-fry cooking of deep-fried foods X. The facilities include, for example, an electric fryer 2 (hereinafter, simply “fryer 2”).

The fryer 2 is a tool equipped with an oil vat 21, a housing 22, and the like.

The oil vat 21 stores frying oil Y therein. The oil vat 21 includes, for example, a handle 30, a fry basket 3, and the like.

The housing 22 accommodates the oil vat 21 therein. On a side surface of the housing 22, switches 22A serving as a setting operation unit for setting the temperature of the frying oil Y, the details of the deep-fry cooking, or the like are provided for each type of the deep-fried foods X.

For deep-frying a food, firstly, a cook places a deep-fried food X before deep-fried into the fry basket 3. Next, the cook hooks the handle 30 on an upper end portion of the housing 22 so that the deep-fried food X before deep-fried is immersed in the frying oil Y. At the same time or around the same time, the cook presses one of the switches 22A which corresponds to the type of the deep-fried food X in cooking.

Upon passage of the time for completing the deep-fry cooking which corresponds to the one of the switches 22A as pressed, the fryer 2 notifies the cook of the completion of deep-frying. At the same time, the fryer 2 causes the fry basket 3 to rise from the oil vat 21 so that the deep-fried food X immersed in the frying oil is pulled up therefrom.

For informing the completion of deep-fry cooking, for example, outputting a buzzer sound from a speaker, displaying the information on a monitor 41 installed on a wall 10A, or the like may be employed. Thus, passage of the time for completing the deep-fry cooking is notified by means of light, sound, or a combination thereof.

The cook who is aware of the completion of deep-fry cooking of the deep-fried food X pulls up the fry basket 3 to take the deep-fried food X out therefrom. Note that the fry basket 3 may be automatically pull up by a drive mechanism.

In the cooking area 1, an imaging device for capturing an image of the frying oil Y is installed. The imaging device is, for example, a video camera 42. Specifically, the video camera 42 is installed to a ceiling 10B.

The video camera 42 captures the surface of the frying oil Y continuously to generate images thereof. The images to be generated are, preferably, in the form of a movie. The video camera 42 is installed with its condition, such as angle of view and focus, being adjusted.

Note that the video camera 42 does not necessarily have to be installed to the ceiling 10B. The video camera 42 may be installed to any position, such as the wall 10A, as long as the position allows the video camera 42 to capture an image of the frying oil Y.

Furthermore, the imaging device does not necessarily have to capture a movie. That is, for example, the imaging device may be a still camera, tablet, or the like for capturing still images. If using a still camera, the one capturing images intermittently along the time series may be adopted.

Furthermore, a plurality of imaging devices may be used. Still further, the imaging device may be a camera or the like equipped in a mobile device such as a tablet or a smartphone.

For example, the learning device 5 is used by being connected to the monitor 41, the video camera 42, and the flyer 2. Note that the learning device 5 may not always be connected to the video camera 42. For example, the learning device 5 may be configured to temporarily store an image captured by the video camera 42 in a storage medium, and separately acquire the image to execute a learning process which will be described later.

### Example of Hardware Configuration of Information Processing Device

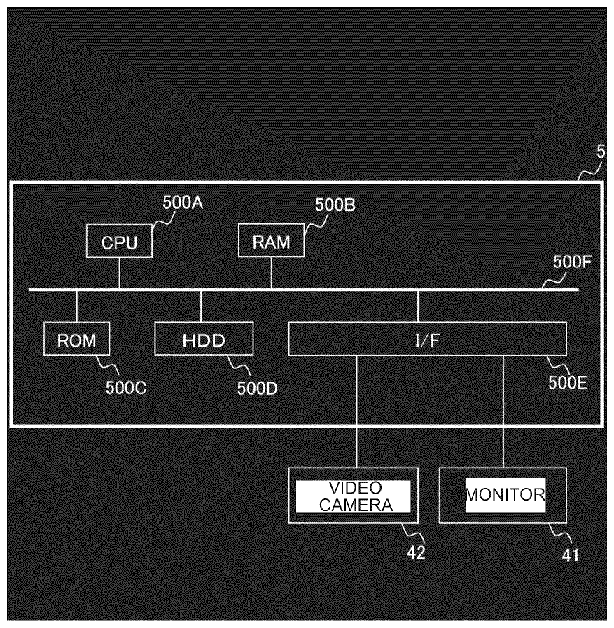

FIG. 2 illustrates an example of a hardware configuration of an information processing device. For example, the learning device 5 is an information processing device configured with the following hardware resources.

The learning device 5 includes a Central Processing Unit (hereinafter, referred to as “CPU 500A”), a Random Access Memory (hereinafter, referred to as “RAM 500B”), and the like. The learning device 5 further includes a Read Only Memory (hereinafter, referred to as “ROM 500C”), a hard disk drive (hereinafter, referred to as “HDD 500D”), interfaces (hereinafter, referred to as “I/F 500E”), and the like.

The CPU 500A is an example of a computing device and a control device.

The RAM 500B is an example of a main storage device.

The ROM 500C and the HDD 500D are examples of secondary storage devices.

The I/F 500E is provided for connection to an input device, an output device, or the like. Specifically, the I/F 500E connects an external device such as the monitor 41 or the video camera 42 by wire or wireless communication for inputting and outputting data.

Note that the hardware configuration of the learning device 5 is not limited to the one described above. For example, the learning device 5 may further include a computing device, a control device, a storage device, an input device, an output device, or a secondary device. Specifically, the information processing device may include a secondary device such as an internal or external Graphics Processing Unit (GPU).

Furthermore, a plurality of learning devices 5 may be provided.

### Example of Entire Processing

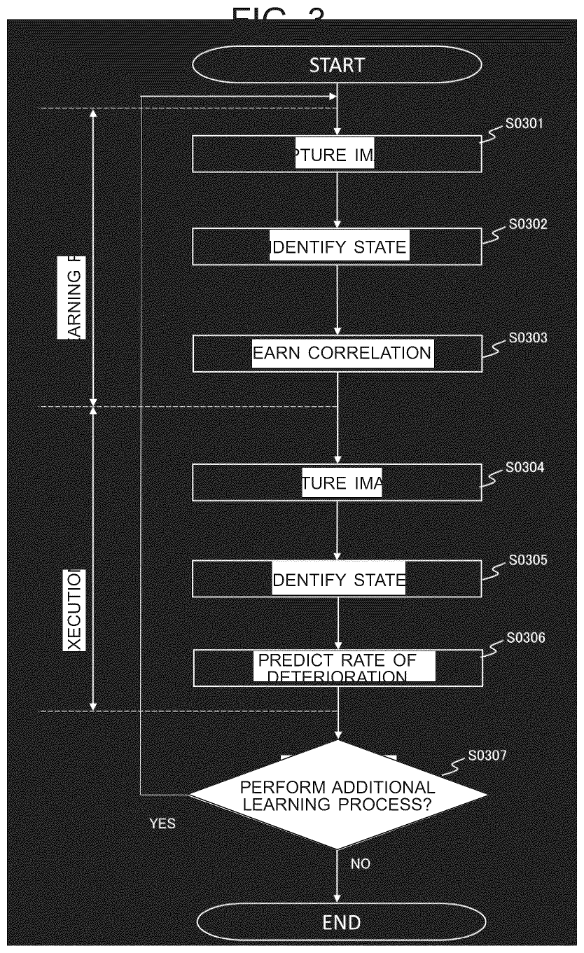

FIG. 3 illustrates an example of entire processing. For example, as illustrated in FIG. 3, the learning device 5 executes the respective processes in the order of “learning process” and “execution process”.

The learning process is the process carried out before the execution process. That is, the learning process is the process of causing a learning model to learn. The execution process is the process using the learned model after learning.

Note that the learning device 5 does not have to execute the learning process and the execution process as consecutive processes as illustrated in FIG. 3. Thus, the learning device 5 may not carry out the execution using the learned model subsequently after the learning of a learning model in the learning process.

For example, the learning device 5 may perform the execution process using the learned model in other opportunities after having created the learned model.

The learning device 5 may start the execution process firstly, using a learned model which has been generated. In the following, an example in which the learning device 5 executes the learning process and the execution process consecutively will be described. That is, Transfer Learning, Fine tuning, or the like may be applied for a learning model and a learned model.

### Example of Learning Process

In step S0301, the learning device 5 captures the edible oil by means of an imaging device or the like to acquire images. Specifically, the images are acquired in the form of a movie or the like.

Preferably, the images are captured, for example, at 15 frames per second (fps) or more. That is, preferably, the images are captured with a temporal resolution equal to or higher than that of the naked eye. On the other hand, the images may be a collection of still images. In a setting with high temporal resolution, bubbles and the like can be accurately recognized.

Preferably, the images are color images. In other words, the images are preferably in a data-format such as RGB or YCrCb. Using color images enables accurate analysis or recognition by colors.

For example, the imaging device is installed so as to capture images of the surface of the edible oil at the center of the captured images. Specifically, the imaging device captures the surface of the edible oil with looking down from a position 40 centimeters or more away from the surface of the edible oil. Based on the optical conditions or the like, the imaging device may be installed at a position other than the position 40 centimeters or more away from the surface of the edible oil.

Note that, the imaging condition may include conditions other than those described above with considering a lighting environment, the size of a fryer, the type of edible oil, or other external environments.

In step S0302, the learning device 5 analyzes an image to identify the state of the edible oil. Firstly, the learning device 5 analyzes the image (a plurality of frames may be used as the image, hereinafter, such a plurality of images is simply referred to as “image” as well) acquired in step S0301.

By analyzing the image, the learning device 5 can recognize, for example, the type of a fried food, the number of pieces, or a combination of them. Specifically, the learning device 5 can recognize various parameters of the subject, such as the type, shape, color, or the number of objects captured in the image by the image recognition process such as pattern matching.

Some or all of these parameters may be acquired via data other than an image or input by a user. For example, the learning device 5 acquires the type of edible oil via, for example, an operation of inputting the name of the edible oil in advance.

The parameters may further include, for example, the amount of edible oil, temperature, or the like. For example, the temperature is acquired by a temperature sensor such as a thermography camera. The amount of the edible oil is acquired by, for example, analysis of an image, a flow meter, a weight measurement device, or the like.

As described above, parameters such as the type of edible oil, type of fried foods, and number of pieces are preferably acquired based on the result of analysis. These parameters are often deeply involved in the tendency of deterioration. Therefore, acquiring these parameters enables accurate AI prediction of deterioration.

A state of edible oil (hereinafter, may be simply referred to as “state”) is preferably indicated by, for example, the rate of formation of bubbles formed per predetermined time in the edible oil, the number of bubbles, the size of a bubble, the speed at which bubbles disappear, or a combination thereof.

For example, by analyzing an image, the learning device 5 can recognize the following bubbles formed on the surface of the edible oil.

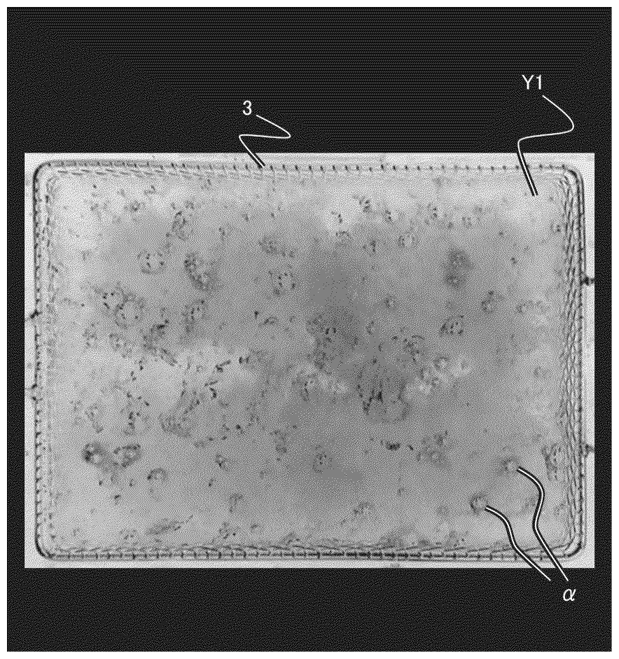

FIG. 4 illustrates an example of bubbles in new edible oil.

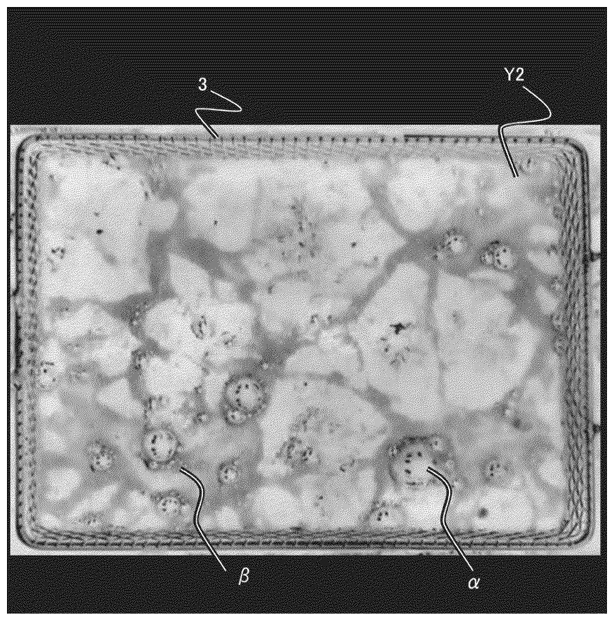

FIG. 5 illustrates an example of bubbles in old edible oil.

For example, an image of edible oil is captured as FIG. 4 or FIG. 5.

FIG. 4 illustrates an example of relatively new edible oil (hereinafter, “edible oil Y1”). On the other hand, FIG. 5 illustrates an example of edible oil (hereinafter, “edible oil Y2”) relatively deteriorated as compared with the edible oil Y1 illustrated in FIG. 4.

For example, as illustrated in FIG. 4 and FIG. 5, during deep-fry cooking, a plurality of bubbles (may be referred to as “surface bubbles”, and hereinafter, simply referred to as “bubbles”) are formed on the surface of edible oil.

These bubbles include, for example, bubbles each having a relatively large diameter (hereinafter, “large bubbles α”) and bubbles each having a relatively small diameter (hereinafter, “fine bubbles β”).

Whether a certain bubble is a large bubble α or a fine bubble β may be classified, for example, by determination based on whether it has a diameter larger than a reference value which has been input in advance.

Specifically, examples of the large bubble α is illustrated in both FIG. 4 and FIG. 5. On the other hand, examples of the fine bubble β are illustrated in FIG. 5.

The fine bubble β may be referred to as “crab bubble”.

The large bubble α has a property of being likely to stay in a position where it is formed. On the other hand, the fine bubble β has a property of being likely to gather with other fine bubbles to form a flow on the surface.

Furthermore, the color of edible oil often darkens and transparency thereof decreases as the edible oil deteriorates. This makes it more difficult to see, in the edible oil Y2, the outline of the fried food X as compared with the edible oil Y1. Thus, the visibility of an outline of the fried food X may be used as an indicator of the rate of deterioration.

Specifically, the learning device 5 analyzes the images to detect the “difference” between the color of the edible oil Y2 and the color of an area of the fried food X. Note that, based on a result of capturing an image of the edible oil Y1, the learning device 5 detects the “difference” between the color of the edible oil Y1 and the color of an area of the fried food X in the edible oil Y1 in advance.

Then, the learning device 5 compares the “difference in color” based on the color of the edible oil Y1 and the color of the edible oil Y2 to estimate the rate of deterioration.

Hereinafter, the large bubble α is referred to as a “first bubble”. On the other hand, the small bubble β is referred to as a “second bubble”. For example, extracting a circle (which may include an ellipse or the like) in the image by the image recognition processing enables recognition of the bubbles including the first bubble and the second bubble.

Then, the learning device 5 recognizes, for example, based on the size of the first bubble, a bubble having a relatively smaller size as the second bubble.

Note that a method of classifying the first bubble and the second bubble is not limited the one described above. For example, whether a certain bubble is the first bubble or the second bubble may be determined based on whether it is larger than a preset reference value. Alternatively, the learning device 5 may recognize the type of a bubble based on a reference other than the size.

As described above, by recognizing the type of a bubble, the learning device 5 can more accurately predict the rate of deterioration.

The rate of formation of bubbles formed per predetermined time can be considered as an example of an indicator of the intensity of foaming on the surface of the edible oil.

Specifically, the rate of formation of bubbles formed per predetermined time is calculated by counting, at the same location on the surface of the frying oil Y, a series of phenomena in which bubbles are formed and then burst as “once”. Accordingly, for example, the rate of formation of bubbles formed per predetermined time in the case where bubbles are formed and then burst at the same location five times is calculated as “five times”.

The number of bubbles is, for example, a statistical value of the number of bubbles in images of several frames. Specifically, the number of bubbles in 30 fps is the average value of the bubbles in 30 frames, i.e., in one second.

The size of a bubble is, for example, a diameter or a circumference of the bubble. Note that the size of a bubble may be a statistical value in images of several frames.

The speed at which bubbles disappear is, for example, the time in which the bubbles are formed until they burst. Note that the speed at which bubbles disappear may be a statistical value of a plurality of bubbles or a statistical value in a predetermined time.

For example, recognizing a bubble in an image by the image recognition processing enables identification of the state. That is, for example, upon extracting an edge component from an image, the learning device 5 recognizes a circle or the like which indicates a bubble, and thus can identify whether a bubble is formed. Note that other methods may be used for recognition of a bubble.

The state may be indicated, not only by the rate of formation of bubbles formed per predetermined time in the edible oil, the number of bubbles, the size of a bubble, the speed at which bubbles disappear, or a combination thereof, but also by other items. That is, the state may be indicated by a result of comprehensive evaluation indicating the state of the edible oil or a qualitative evaluation.

Furthermore, the state may be evaluated by converting a value into a value per one piece of fried food. For example, for evaluating five pieces of fried food, firstly, the state such as the rate of formation of bubbles formed per predetermined time in the edible oil is measured as a whole. Thereafter, the state is calculated by dividing the result of total measurement by the number of pieces “five”.

As described above, the state may be measured by converting the whole state in terms of one piece. However, the size, weight, and the like of each piece of fried food are not always uniform. Accordingly, in calculating a value per one piece of fried food, the value for whole may be divided by values other than the number of fried foods (in this example, “five”).

In particular, for evaluating the rate of formation of bubbles formed per predetermined time in the edible oil and the number of bubbles, values as obtained are preferably converted into a value per one piece of fried food.

In step S0303, the learning device 5 causes the learning model to learn correlations.

For example, the learning device 5 inputs, for each state identified in step S0302, the rate of deterioration corresponding thereto. That is, the learning device 5 inputs the rate of deterioration as “correct answer data” in the learning. Note that the rate of deterioration may be estimated based on analysis of an image, use time of the edible oil, or the like.

The rate of deterioration is, for example, an acid value (AV) of edible oil. However, the rate of deterioration may be indicated by the rate of increase in viscosity of edible oil, the color of edible oil, an Anisidine value of edible oil, the quantity of polar compounds of edible oil, a Carbonyl value of edible oil, a smoke point of edible oil, the Tocopherol content of edible oil, an iodine value of edible oil, a refractive index of edible oil, the quantity of volatile compounds of edible oil, the composition of volatile compounds of edible oil, the flavor of edible oil, the quantity of volatile compounds of a deep-fried food obtained by deep-fry cooking with edible oil, the composition of volatile compounds of a deep-fried food, the flavor of a deep-fried food, or a combination thereof.

An acid value (may be referred to as “AV”) of edible oil is a value measured by, for example, the standard methods for the analysis of fats, oils and related materials, 2.3.1-2013.

A rate of increase in viscosity of edible oil is, for example, a value calculated using the ratio of the amount of increase in viscosity relative to a reference value that is, for example, the viscosity of new edible oil before being used in deep-fry cooking for the first time after changing of oil (that is, viscosity at the start of use). Note that the viscosity is measured by a viscometer or the like. The viscometer is, for example, an E-type viscometer (TVE-25H, made by Toki Sangyo Co., Ltd.).

The color of edible oil (may be referred to as “color tone” or “hue”) is a value measured by, for example, the standard methods for the analysis of fats, oils and related materials, 2.2.1.1-2013. (for example, using a yellow component value and a red component value, a value is calculated by “the yellow component value plus 10×the red component value”).

An Anisidine value of edible oil is a value measured by the standard methods for the analysis of fats, oils and related materials, 2.5.3-2013.

The quantity of polar compounds of edible oil is a value measured by the standard methods for the analysis of fats, oils and related materials, 2.5.5-2013. For example, the quantity of polar compounds of edible oil is a value measured by a polar compound measurement device (such as the one made by Testo K.K.).

A Carbonyl value of edible oil is a value measured by, for example, the standard methods for the analysis of fats, oils and related materials, 2.5.4.2-2013.

A smoke point of edible oil is a value measured by the standard methods for the analysis of fats, oils and related materials, 2.2.11.1-2013. Smoke generates due to combustion of lipids contained in edible oil or decomposed products thereof.

The Tocopherol content of edible oil (may referred to as “vitamin E”) is the content of Tocopherol contained in the edible oil. Tocopherol is a value measured by, for example, a High Performance Liquid Chromatography (HPLC) method.

An iodine value of edible oil indicates, for example, the grams of iodine that can be added to 100 grams of oil or fat. An iodine value of edible oil is a value measured by, for example, the standard methods for the analysis of fats, oils and related materials, 2.3.41-2013.

A refractive index of edible oil is a value measured by, for example, the standard methods for the analysis of fats, oils and related materials, 2.2.3-2013.

The quantity of volatile compounds of edible oil, the composition of volatile compounds of edible oil, the quantity of volatile compounds of a deep-fried food obtained by deep-fry cooking with edible oil, and the composition of volatile compounds of a deep-fried food are defined by components (mainly odor components) volatilized from the deep-fried food or the edible oil. In the edible oil, the quantity or composition of the volatile components changes with deterioration of the edible oil. The volatile components may be measured by, for example, a Gas Chromatograph-Mass Spectrometer (GC-MS) or an odor sensor.

The flavor of edible oil and the flavor of a deep-fried food are values measured by sensory evaluation (for example, evaluation by a person who has actually eaten the deep-fried food) or a taste sensor.

As an indicator of the rate of deterioration of edible oil, not only a chemical one but also, for example, a criterion such as deliciousness may be employed.

For example, upon estimating a state as described above and the rate of deterioration based on input or analysis of an image, the learning device 5 can cause the learning model to learn the correlation between the state and the rate of deterioration. Repeating such learning processes causes the learning model to learn, whereby a learned model for predicting the rate of deterioration is generated in the execution processing thereafter.

Hereinafter, an example in which the learning device 5 carries out the execution process using the learned model, that is, the prediction device is the learning device 5 will be described. However, the prediction device may be an information processing device other than the learning device 5.

Note that the learning device 5 may carry out an additional learning process after the execution process. For example, when adding and further carrying out the learning process after the execution process, the learning device 5 determines whether to add and carry out the learning process in step S0307 or the like.

### Example of Execution Process

For example, the learning device 5 carries out the execution process as follows to predict the rate of deterioration.

In step S0304, the learning device 5 causes an imaging device to capture an image of the edible oil to acquire the image. For example, the process of step S0304 is the same as that of step S0301.

In step S0305, the learning device 5 analyzes the image to identify the state of the edible oil. For example, the process of step S0305 is the same as that of step S0302. Thus, in step S0305, the learning device 5 acquires a parameter to be used for prediction.

In step S0306, the learning device 5 predicts the rate of deterioration. Specifically, the learning device 5 outputs the rate of deterioration of the edible oil indicated in the image. Note that, based on the rate of deterioration, the learning device 5 may output the tendency of deterioration or the information in the format in which when to change the edible oil, such as whether it is time to change the edible oil, is predicted.

In step S0307, the learning device 5 determines whether to perform an additional learning process. For example, when determination that it is necessary to further improve the accuracy as a result of the execution process, the learning device 5 carries out the additional learning process.

Note that whether to carry out the additional learning process may be determined at timing other than the one described above. For example, whether to carry out the additional learning process may be determined before the execution process, after a plurality of execution processes, or the like. The additional learning process determined to be performed may be carried out by an information processing device other than the learning device 5.

When carrying out the additional learning process (YES in step S0307), the learning device 5 returns to step S0301, that is, adds and carries out the learning process. On the other hand, when not carrying out the additional learning process (NO in step S0307), the learning device 5 ends the entire process.

The results of the following experiments show that there is a correlation between each state and the rate of deterioration.

### Details and Results of Experiments

In the experiments below, six types of fried food were used as samples. In the following, these samples are referred to as “sample A”, “sample B”, “sample C”, “sample D”, “sample E”, and “sample F”. The experiments were performed under the conditions described below.
Edible oil to be used: Rapeseed oil
Temperature of oil in deep-fry cooking: 180 degrees Celsius
Time of deep-fry cooking: 5 minutes
Number of fried foods: 4 pieces/time

Evaluation method: Sensory evaluation is performed by two evaluators (where the number of samples is n=2) over four periods of time in which 5 minutes from the start of use of the edible oil are divided. In the four periods of time of five minutes, “0 (minutes): 00 (seconds) to 0:20” is referred to as a “first period”. In the same manner, in the five minutes, “0:40 to 1:00” is referred to as a “second period”, “2:20 to 2:40” is referred to as a “third period”, and “4:40 to 5:00” is referred to as a “fourth period”.

The results of experiments will be described below for each period and evaluation item.

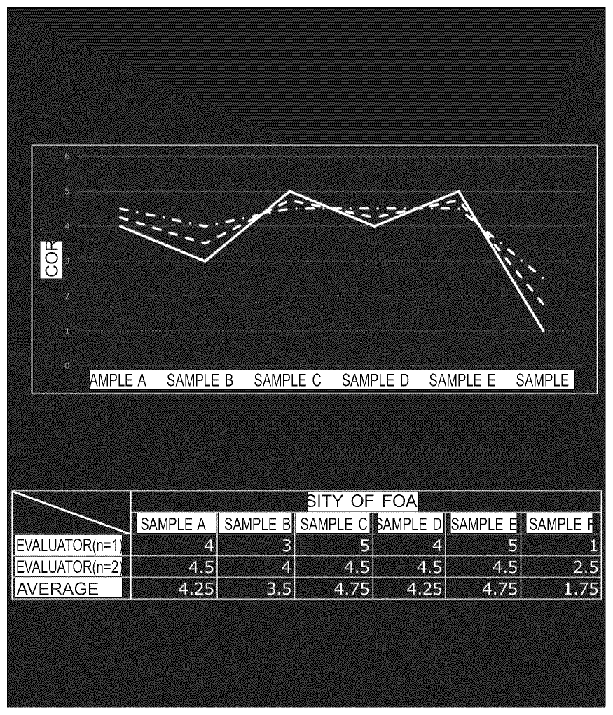

FIG. 6 illustrates the result of experiment of “intensity of foaming” in the first period. The graph shows the results of evaluation of “intensity of foaming” for each sample by two evaluators.

In the graph, the evaluation result by the evaluator of the first person (referred to as “n=1”) is illustrated by a solid line, the evaluation result by the evaluator of the second person (referred to as “n=2”) is illustrated by an alternate long and short dash line, and the average of the evaluation results of the two evaluators is illustrated by a dotted line.

The horizontal axis represents the type of sample while the vertical axis represents the result of evaluation. The result of evaluation is indicated with the score from “1” to “5”. The higher the value of the score is, the greater the “foaming intensity” is. In the following description, the same applies to the other experiments.

In the evaluation item, when the graph of each evaluator has a waveform similar to those of the other graphs, it is found that there is less variation depending on the evaluators.

The average value of the evaluation values of “Sample A” was “4.25”.

The average value of the evaluation values of “Sample B” was “3.5”.

The average value of the evaluation values of “Sample C” was “4.75”.

The average value of the evaluation values of “Sample D” was “4.25”.

The average value of the evaluation values of “Sample E” was “4.75”.

The average value of the evaluation values of “Sample F” was “1.75”.

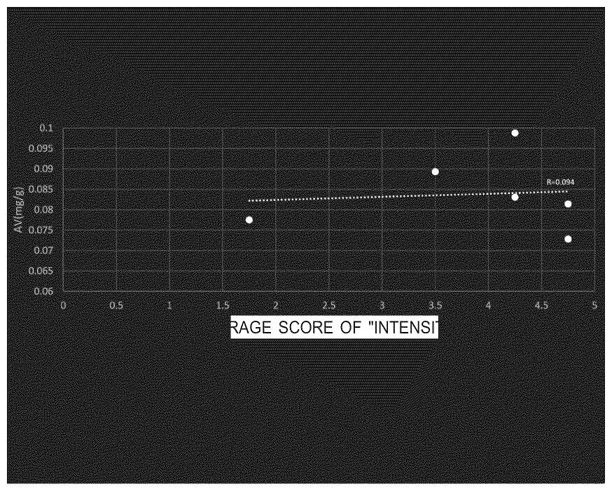

FIG. 7 illustrates a correlation between the “intensity of foaming” and acid values in the first period. FIG. 7 illustrates a graph obtained by plotting the results of evaluation by the experiment depicted in FIG. 6 (hereinafter, average values are used as representative values).

The horizontal axis represents values of the representative values while the vertical axis represents acid values (AV).

The graph shows the result of single regression analysis. The correlation is expressed with a correlation coefficient of “R”. Accordingly, the closer “R” is to “1”, the stronger the correlation between the two variables is.

In this experiment, “R=0.094” was obtained.

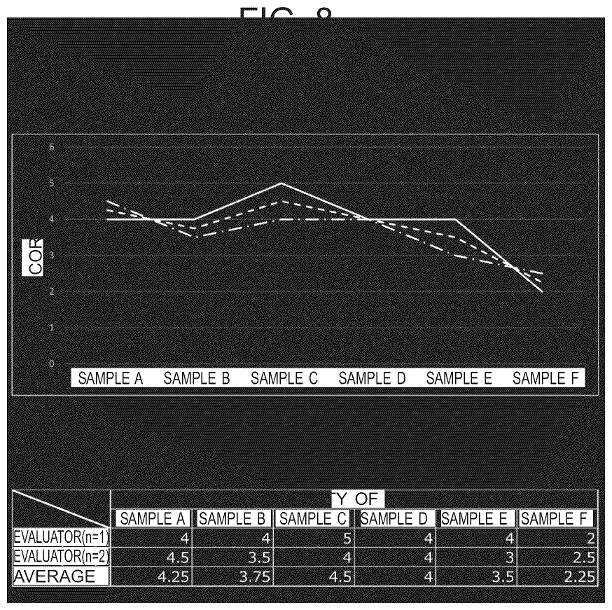

FIG. 8 illustrates the results of experiment of “quantity of form” in the first period. The experiment shown in FIG. 8 has the evaluation item different from that of FIG. 6. In the following, explanation for the issues common to those in FIG. 6 will be omitted.

The average value of the evaluation values of “Sample A” was “4.25”.

The average value of the evaluation values of “Sample B” was “3.75”.

The average value of the evaluation values of “Sample C” was “4.5”.

The average value of the evaluation values of “Sample D” was “4”.

The average value of the evaluation values of “Sample E” was “3.5”.

The average value of the evaluation values of “Sample F” was “2.25”.

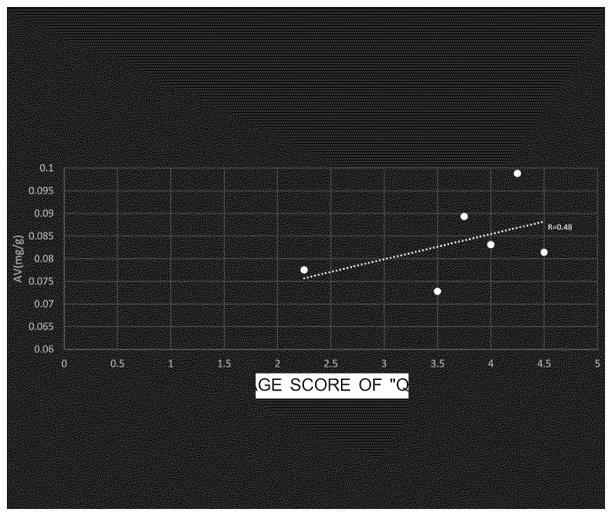

FIG. 9 illustrates the correlation between the “quantity of form” and acid values in the first period. The experiment shown in FIG. 9 has the evaluation item different from that of FIG. 6. In the following, explanation for the issues common to those in FIG. 6 will be omitted.

In this experiment, “R=0.48” was obtained.

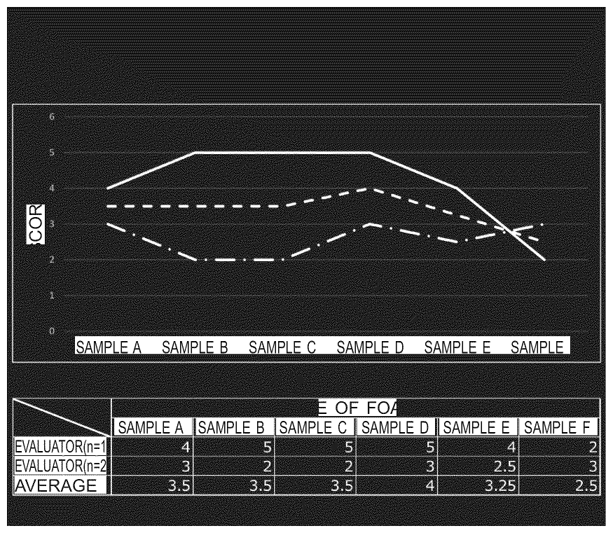

FIG. 10 illustrates the results of experiment of “size of foam” in the first period. The experiment shown in FIG. 10 has the evaluation item different from that of FIG. 6. In the following, explanation for the issues common to those in FIG. 6 will be omitted.

The average value of the evaluation values of “Sample A” was “3.5”.

The average value of the evaluation values of “Sample B” was “3.5”.

The average value of the evaluation values of “Sample C” was “3.5”.

The average value of the evaluation values of “Sample D” was “4”.

The average value of the evaluation values of “Sample E” was “3.25”.

The average value of the evaluation values of “Sample F” was “2.5”.

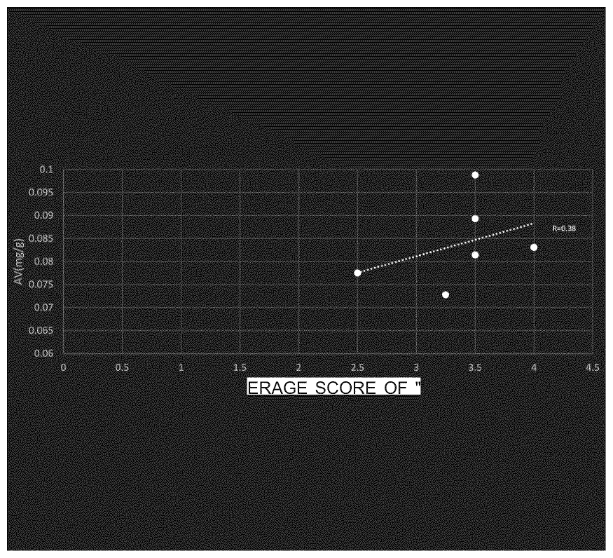

FIG. 11 illustrates the correlation between the “size of foam” and acid values in the first period. The experiment shown in FIG. 11 has the evaluation item different from that of FIG. 7. In the following, explanation for the issues common to those in FIG. 7 will be omitted.

In this experiment, “R=0.38” was obtained.

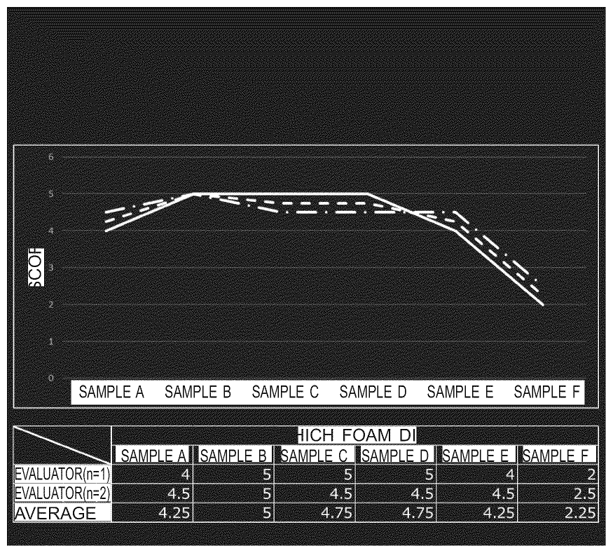

FIG. 12 illustrates the results of experiment of “speed at which foam disappears” in the first period. The experiment shown in FIG. 12 has the evaluation item different from that of FIG. 6. In the following, explanation for the issues common to those in FIG. 6 will be omitted.

The average value of the evaluation values of “Sample A” was “4.25”.

The average value of the evaluation values of “Sample B” was “5”.

The average value of the evaluation values of “Sample C” was “4.75”.

The average value of the evaluation values of “Sample D” was “4.75”.

The average value of the evaluation values of “Sample E” was “4.25”.

The average value of the evaluation values of “Sample F” was “2.25”.

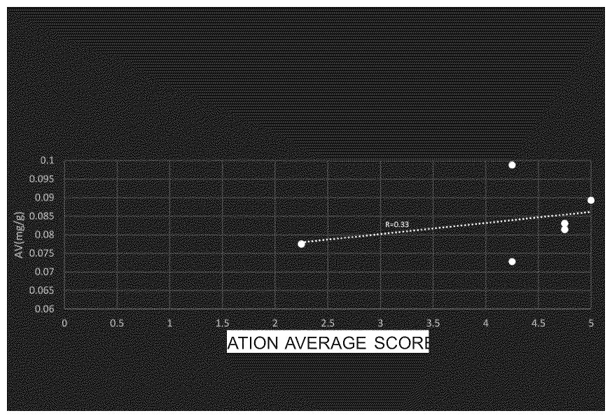

FIG. 13 illustrates the correlation between the “speed at which foam disappears” and acid values in the first period. The experiment shown in FIG. 13 has the evaluation item different from that of FIG. 7. In the following, explanation for the issues common to those in FIG. 7 will be omitted.

In this experiment, “R=0.33” was obtained.

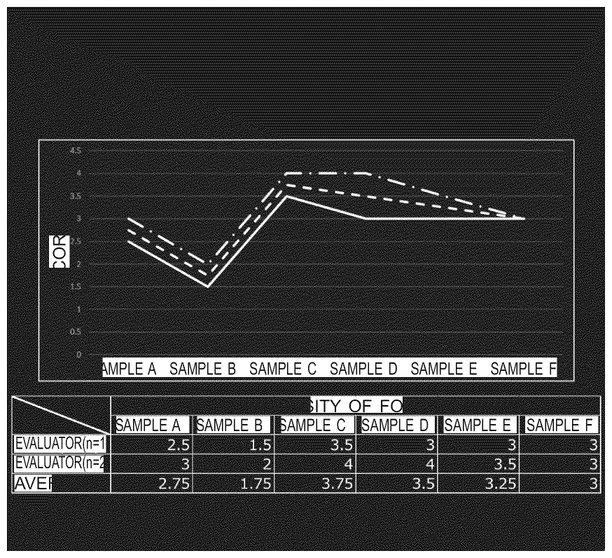

FIG. 14 illustrates the results of experiment of “intensity of foaming” in the second period. The experiment shown in FIG. 14 differs from FIG. 6 in the periods. In the following, explanation for the issues common to those in FIG. 6 will be omitted.

The average value of the evaluation values of “Sample A” was “2.75”.

The average value of the evaluation values of “Sample B” was “1.75”.

The average value of the evaluation values of “Sample C” was “3.75”.

The average value of the evaluation values of “Sample D” was “3.5”.

The average value of the evaluation values of “Sample E” was “3.25”.

The average value of the evaluation values of “Sample F” was “3”.

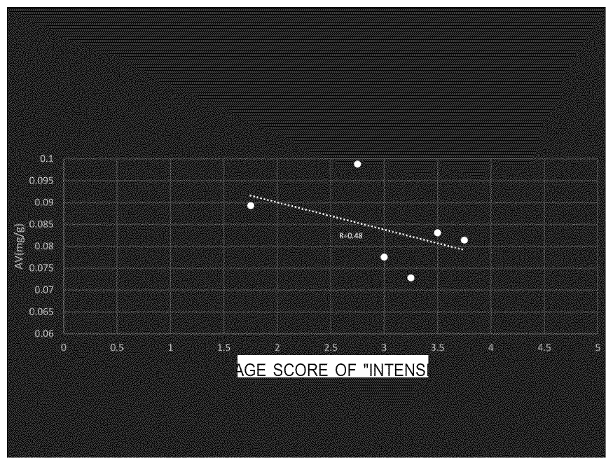

FIG. 15 illustrates the correlation between the “intensity of foaming” and acid values in the second period. The experiment shown in FIG. 15 has the evaluation item different from that of FIG. 7. In the following, explanation for the issues common to those in FIG. 7 will be omitted.

In this experiment, “R=0.48” was obtained.

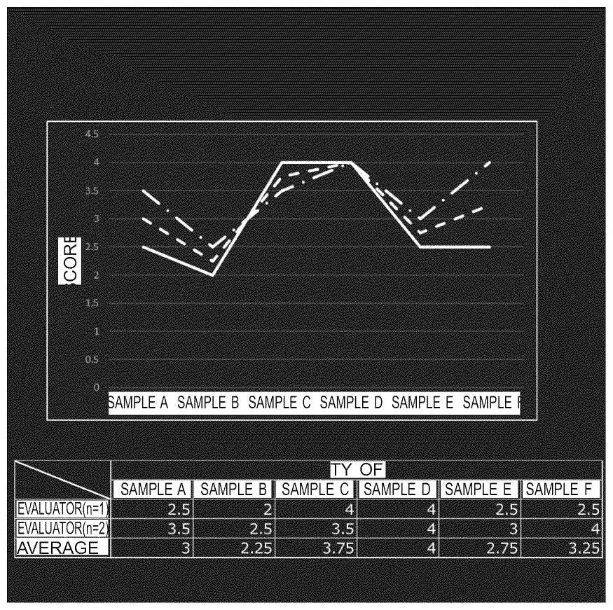

FIG. 16 illustrates the results of “quantity of form” in the second period. The experiment shown in FIG. 16 has the evaluation item different from that of FIG. 14. In the following, explanation for the issues common to those in FIG. 14 will be omitted.

The average value of the evaluation values of “Sample A” was “3”.

The average value of the evaluation values of “Sample B” was “2.25”.

The average value of the evaluation values of “Sample C” was “3.75”.

The average value of the evaluation values of “Sample D” was “4”.

The average value of the evaluation values of “Sample E” was “2.75”.

The average value of the evaluation values of “Sample F” was “3.25”.

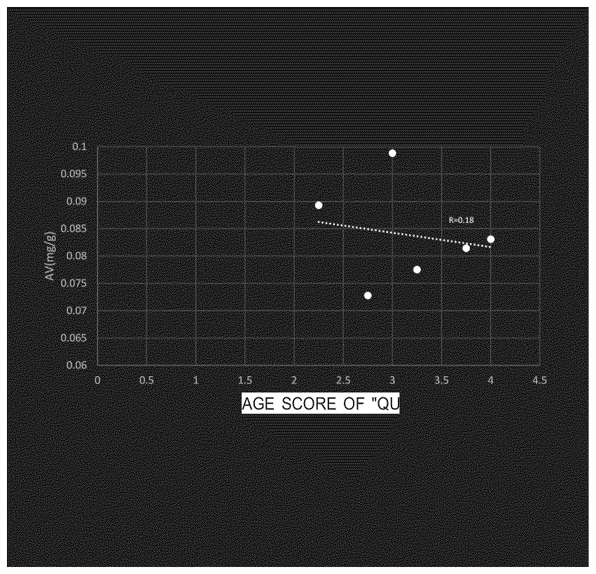

FIG. 17 illustrates the correlation between the “quantity of form” and acid values in the second period. The experiment shown in FIG. 17 has the evaluation item different from that of FIG. 15. In the following, explanation for the issues common to those in FIG. 15 will be omitted.

In this experiment, “R=0.18” was obtained.

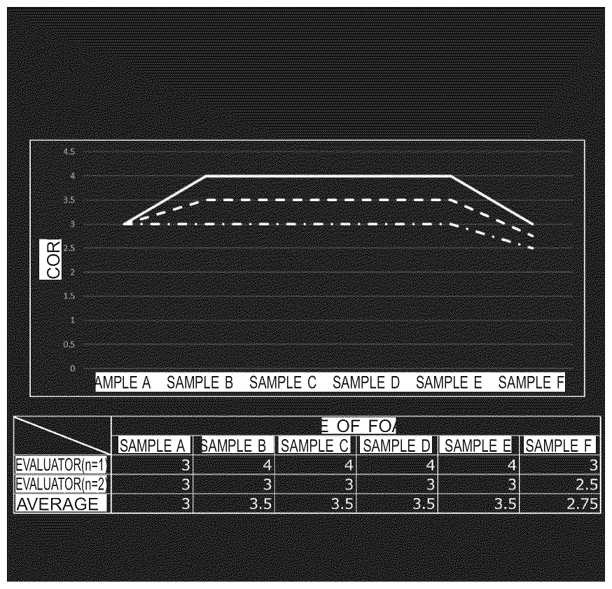

FIG. 18 illustrates the results of “size of foam” in the second period. The experiment shown in FIG. 18 has the evaluation item different from that of FIG. 14. In the following, explanation for the issues common to those in FIG. 14 will be omitted.

The average value of the evaluation values of “Sample A” was “3”.

The average value of the evaluation values of “Sample B” was “3.5”.

The average value of the evaluation values of “Sample C” was “3.5”.

The average value of the evaluation values of “Sample D” was “3.5”.

The average value of the evaluation values of “Sample E” was “3.5”.

The average value of the evaluation values of “Sample F” was “2.75”.

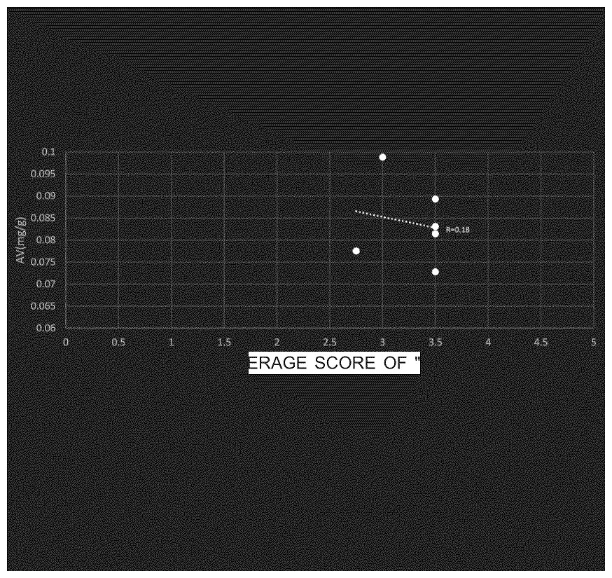

FIG. 19 illustrates the correlation between the “size of foam” and acid values in the second period. The experiment shown in FIG. 19 has the evaluation item different from that of FIG. 15. In the following, explanation for the issues common to those in FIG. 15 will be omitted.

In this experiment, “R=0.18” was obtained.

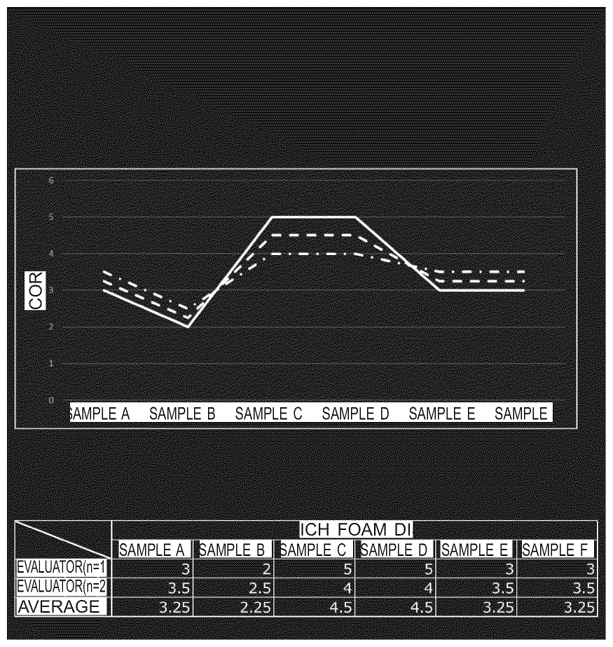

FIG. 20 illustrates the results of “speed at which foam disappears” in the second period. The experiment shown in FIG. 20 has the evaluation item different from that of FIG. 14. In the following, explanation for the issues common to those in FIG. 14 will be omitted.

The average value of the evaluation values of “Sample A” was “3.25”.

The average value of the evaluation values of “Sample B” was “2.25”.

The average value of the evaluation values of “Sample C” was “4.5”.

The average value of the evaluation values of “Sample D” was “4.5”.

The average value of the evaluation values of “Sample E” was “3.25”.

The average value of the evaluation values of “Sample F” was “3.25”.

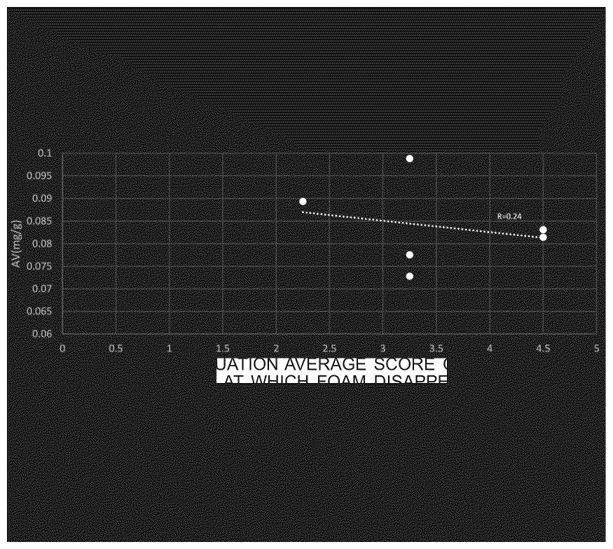

FIG. 21 illustrates the correlation between the “speed at which foam disappears” and acid values in the second period. The experiment shown in FIG. 21 has the evaluation item different from that of FIG. 15. In the following, explanation for the issues common to those in FIG. 15 will be omitted.

In this experiment, “R=0.24” was obtained.

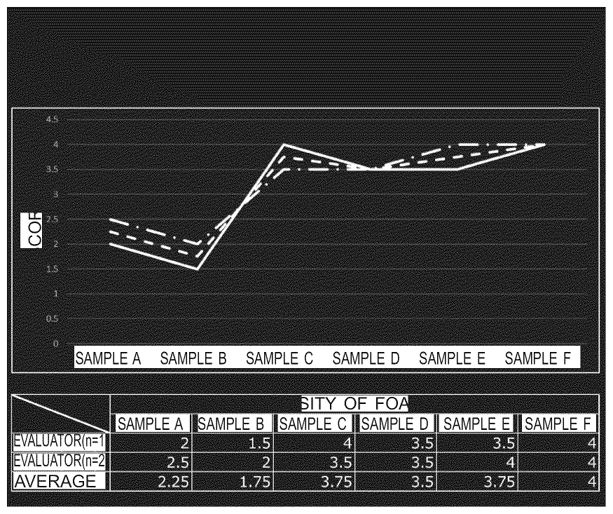

FIG. 22 illustrates the results of experiment of “intensity of foaming” in the third period. The experiment shown in FIG. 22 differs from FIG. 6 in the periods. In the following, explanation for the issues common to those in FIG. 6 will be omitted.

The average value of the evaluation values of “Sample A” was “2.25”.

The average value of the evaluation values of “Sample B” was “1.75”.

The average value of the evaluation values of “Sample C” was “3.75”.

The average value of the evaluation values of “Sample D” was “3.5”.

The average value of the evaluation values of “Sample E” was “3.75”.

The average value of the evaluation values of “Sample F” was “4”.

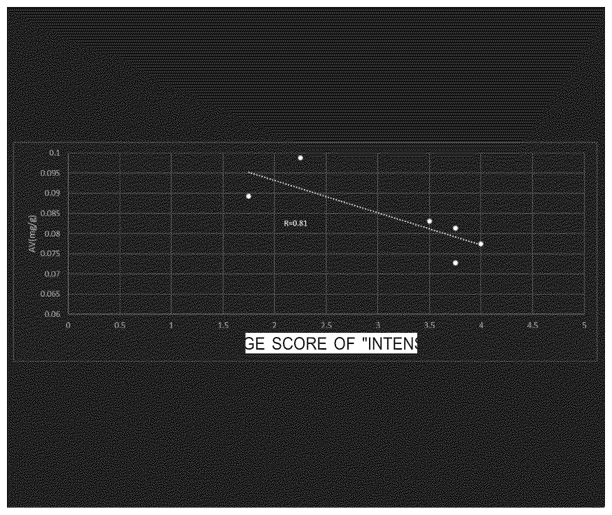

FIG. 23 illustrates the correlation between the “intensity of foaming” and acid values in the third period. The experiment shown in FIG. 23 has the evaluation item different from that of FIG. 7. In the following, explanation for the issues common to those in FIG. 7 will be omitted.

In this experiment, “R=0.81” was obtained.

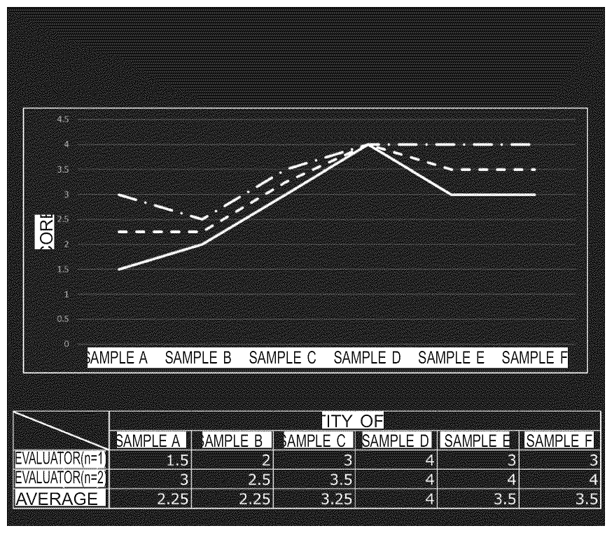

FIG. 24 illustrates the results of experiment of “quantity of form” in the third period. The experiment shown in FIG. 24 has the evaluation item different from that of FIG. 22. In the following, explanation for the issues common to those in FIG. 22 will be omitted.

The average value of the evaluation values of “Sample A” was “2.25”.

The average value of the evaluation values of “Sample B” was “2.25”.

The average value of the evaluation values of “Sample C” was “3.25”.

The average value of the evaluation values of “Sample D” was “4”.

The average value of the evaluation values of “Sample E” was “3.5”.

The average value of the evaluation values of “Sample F” was “3.5”.

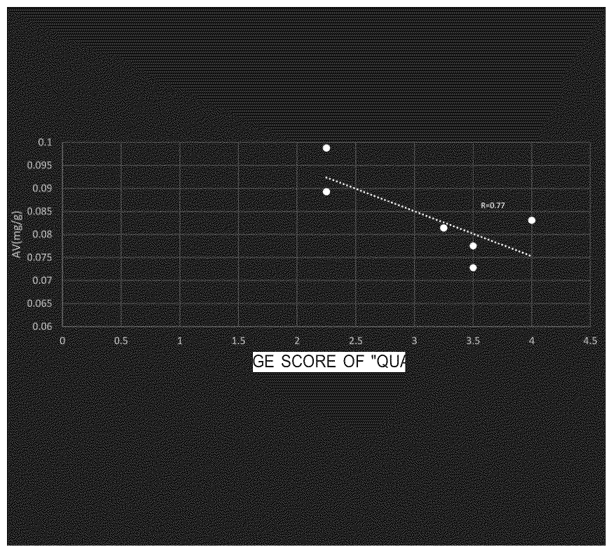

FIG. 25 illustrates the correlation between the “quantity of form” and acid values in the third period. The experiment shown in FIG. 25 has the evaluation item different from that of FIG. 23. In the following, explanation for the issues common to those in FIG. 23 will be omitted.

In this experiment, “R=0.77” was obtained.

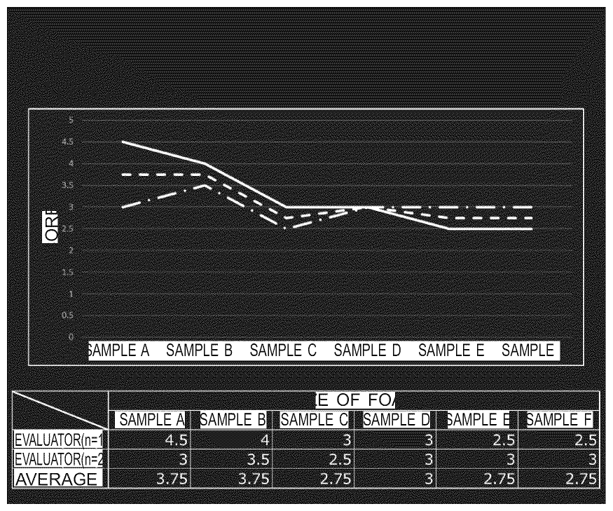

FIG. 26 illustrates the results of experiment of “size of foam” in the third period. The experiment shown in FIG. 26 has the evaluation item different from that of FIG. 22. In the following, explanation for the issues common to those in FIG. 22 will be omitted.

The average value of the evaluation values of “Sample A” was “3.75”.

The average value of the evaluation values of “Sample B” was “3.75”.

The average value of the evaluation values of “Sample C” was “2.75”.

The average value of the evaluation values of “Sample D” was “3”.

The average value of the evaluation values of “Sample E” was “2.75”.

The average value of the evaluation values of “Sample F” was “2.75”.

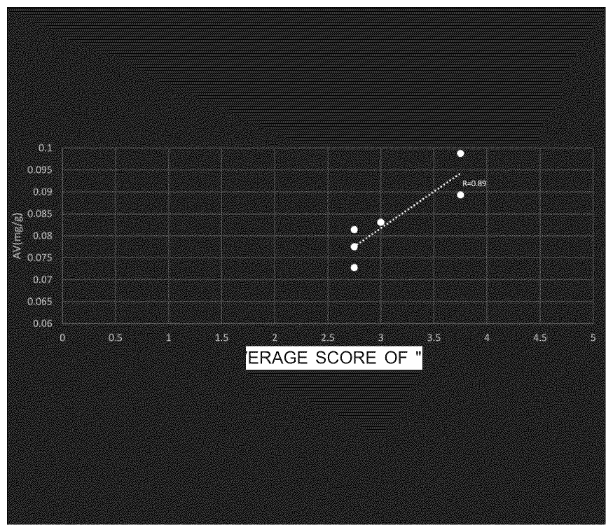

FIG. 27 illustrates the correlation between the “size of foam” and acid values in the third period. The experiment shown in FIG. 27 has the evaluation item different from that of FIG. 23. In the following, explanation for the issues common to those in FIG. 23 will be omitted.

In this experiment, “R=0.89” was obtained.

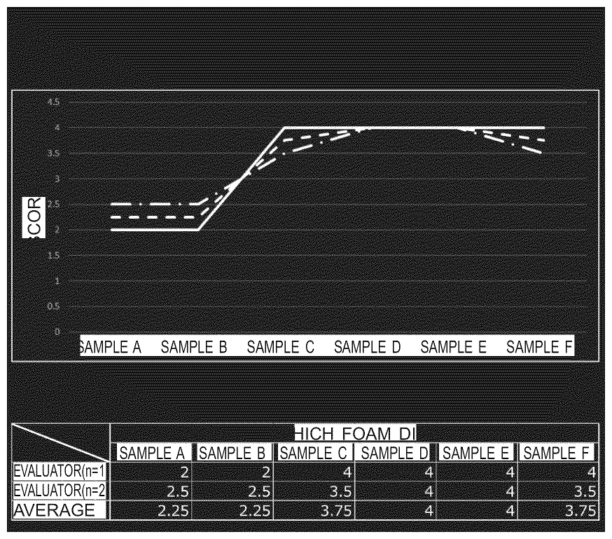

FIG. 28 illustrates the results of experiment of “speed at which foam disappears” in the third period. The experiment shown in FIG. 28 has the evaluation item different from that of FIG. 22. In the following, explanation for the issues common to those in FIG. 22 will be omitted.

The average value of the evaluation values of “Sample A” was “2.25”.

The average value of the evaluation values of “Sample B” was “2.25”.

The average value of the evaluation values of “Sample C” was “3.75”.

The average value of the evaluation values of “Sample D” was “4”.

The average value of the evaluation values of “Sample E” was “4”.

The average value of the evaluation values of “Sample F” was “3.75”.

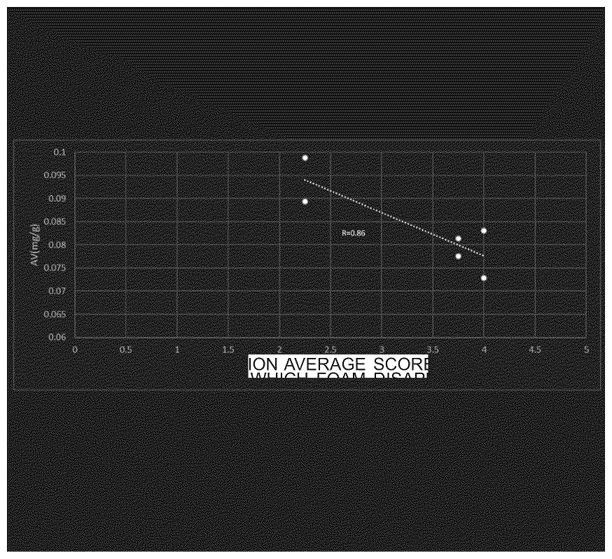

FIG. 29 illustrates the correlation between the “speed at which foam disappears” and acid values in the third period. The experiment shown in FIG. 29 has the evaluation item different from that of FIG. 23. In the following, explanation for the issues common to those in FIG. 23 will be omitted.

In this experiment, “R=0.86” was obtained.

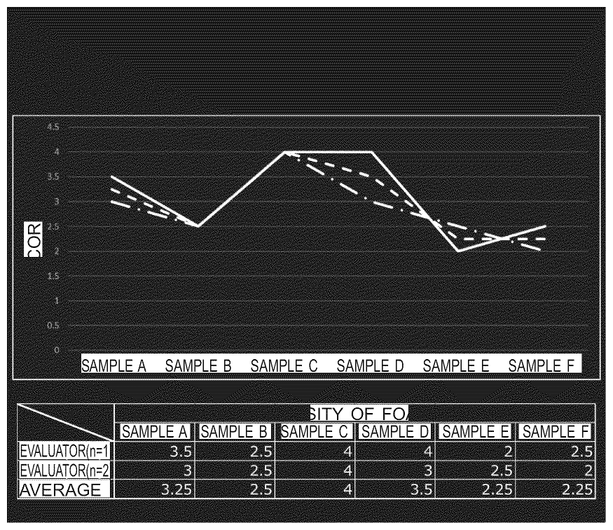

FIG. 30 illustrates the results of experiment of “intensity of foaming” in the fourth period. The experiment shown in FIG. 30 differs from FIG. 6 in the periods. In the following, explanation for the issues common to those in FIG. 6 will be omitted.

The average value of the evaluation values of “Sample A” was “3.25”.

The average value of the evaluation values of “Sample B” was “2.5”.

The average value of the evaluation values of “Sample C” was “4”.

The average value of the evaluation values of “Sample D” was “3.5”.

The average value of the evaluation values of “Sample E” was “2.25”.

The average value of the evaluation values of “Sample F” was “2.25”.

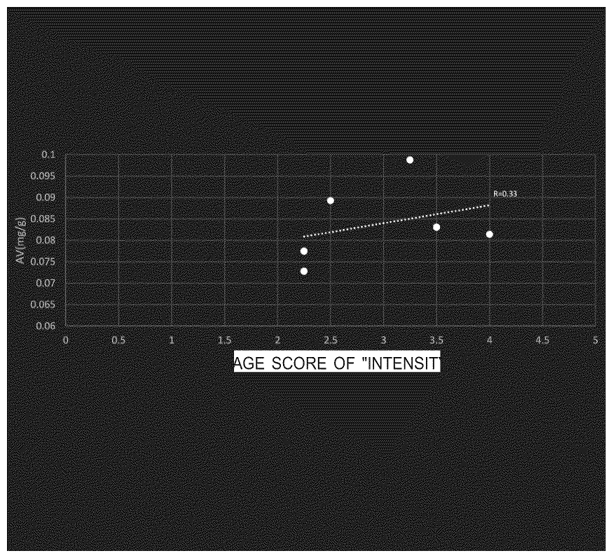

FIG. 31 illustrates the correlation between the “intensity of foaming” and acid values in the fourth period. The experiment shown in FIG. 31 has the evaluation item different from that of FIG. 7. In the following, explanation for the issues common to those in FIG. 7 will be omitted.

In this experiment, “R=0.33” was obtained.

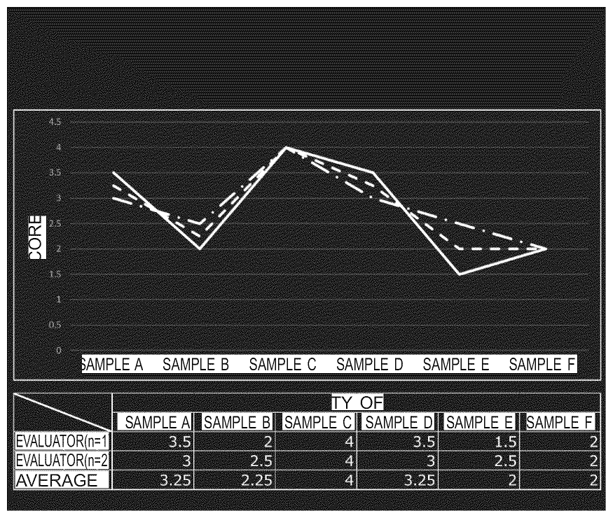

FIG. 32 illustrates the results of “quantity of form” in the fourth period. The experiment shown in FIG. 32 has the evaluation item different from that of FIG. 30. In the following, explanation for the issues common to those in FIG. 30 will be omitted.

The average value of the evaluation values of “Sample A” was “3.25”.

The average value of the evaluation values of “Sample B” was “2.25”.

The average value of the evaluation values of “Sample C” was “4”.

The average value of the evaluation values of “Sample D” was “3.25”.

The average value of the evaluation values of “Sample E” was “2”.

The average value of the evaluation values of “Sample F” was “2”.

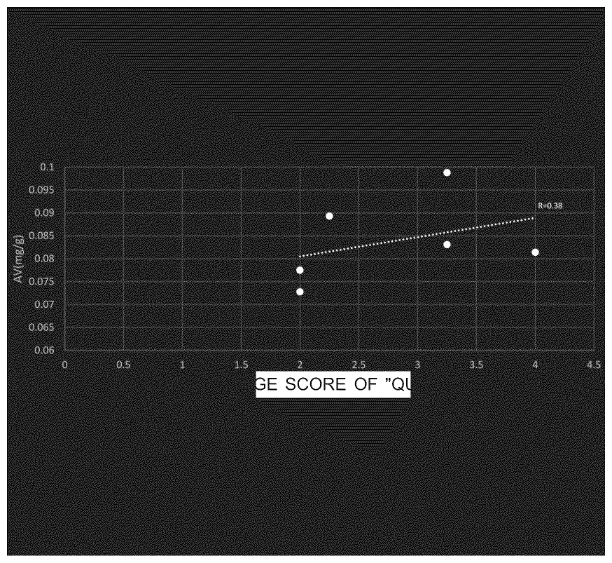

FIG. 33 illustrates the correlation between the “quantity of form” and acid values in the fourth period. The experiment shown in FIG. 33 has the evaluation item different from that of FIG. 31. In the following, explanation for the issues common to those in FIG. 31 will be omitted.

In this experiment, “R=0.38” was obtained.

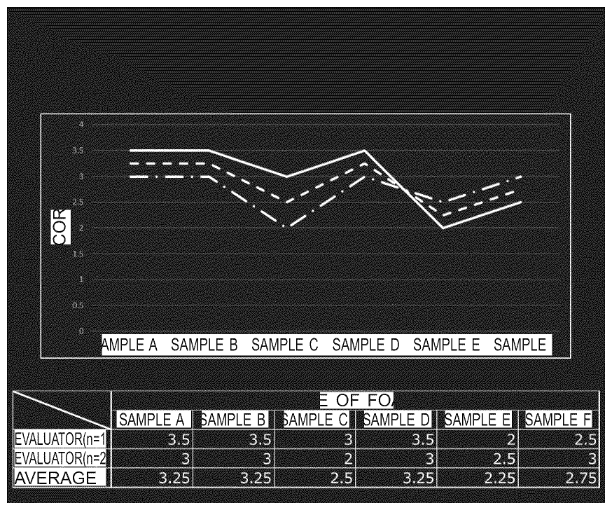

FIG. 34 illustrates the results of “quantity of form” in the fourth period. The experiment shown in FIG. 34 has the evaluation item different from that of FIG. 30. In the following, explanation for the issues common to those in FIG. 30 will be omitted.

The average value of the evaluation values of “Sample A” was “3.25”.

The average value of the evaluation values of “Sample B” was “3.25”.

The average value of the evaluation values of “Sample C” was “2.5”.

The average value of the evaluation values of “Sample D” was “3.25”.

The average value of the evaluation values of “Sample

E” was “2.25”.

The average value of the evaluation values of “Sample F” was “2.75”.

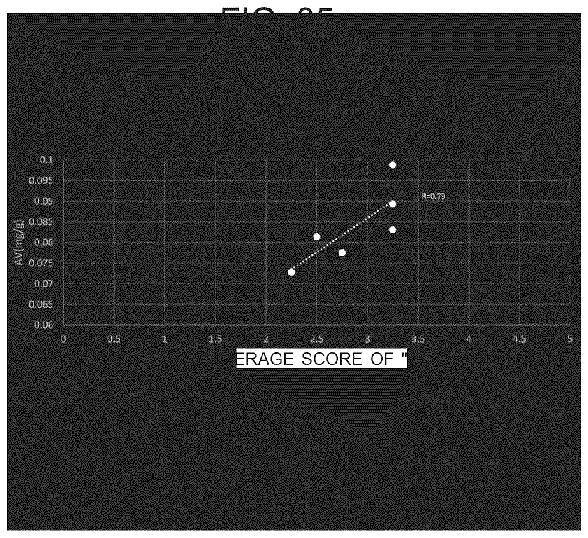

FIG. 35 illustrates the correlation between the “size of foam” and acid values in the fourth period. The experiment shown in FIG. 35 has the evaluation item different from that of FIG. 31. In the following, explanation for the issues common to those in FIG. 31 will be omitted.

In this experiment, “R=0.79” was obtained.

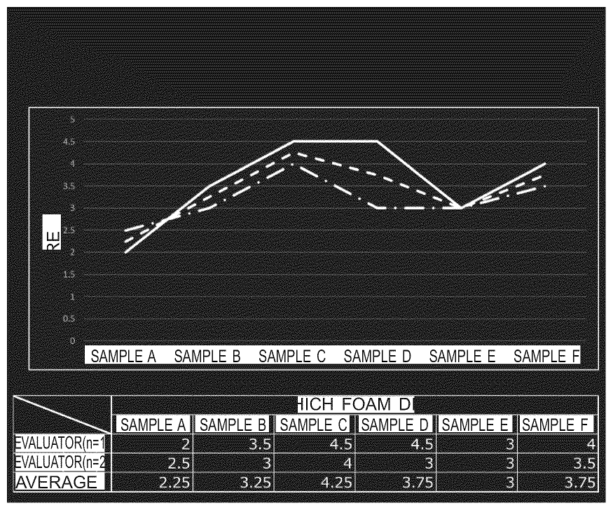

FIG. 36 illustrates the results of “speed at which foam disappears” in the fourth period. The experiment shown in FIG. 36 has the evaluation item different from that of FIG. 30. In the following, explanation for the issues common to those in FIG. 30 will be omitted.

The average value of the evaluation values of “Sample A” was “2.25”.

The average value of the evaluation values of “Sample B” was “3.25”.

The average value of the evaluation values of “Sample C” was “4.25”.

The average value of the evaluation values of “Sample D” was “3.75”.

The average value of the evaluation values of “Sample E” was “3”.

The average value of the evaluation values of “Sample F” was “3.75”.

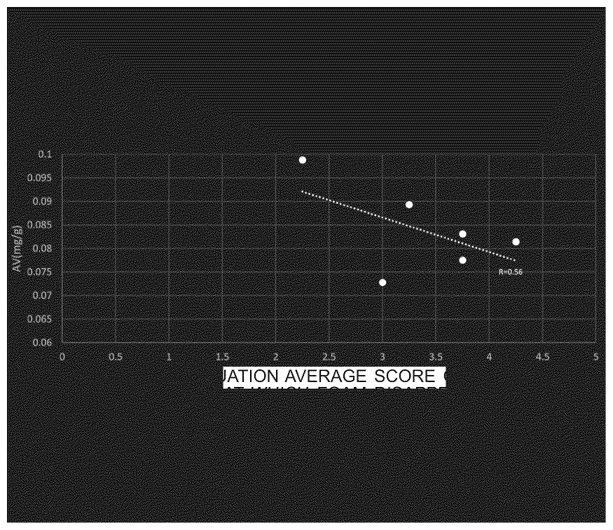

FIG. 37 illustrates the correlation between the “speed at which foam disappears” and acid values in the fourth period. The experiment shown in FIG. 37 has the evaluation item different from that of FIG. 31. In the following, explanation for the issues common to those in FIG. 31 will be omitted.

In this experiment, “R=0.56” was obtained.

The evaluation results of, for example, the “intensity of foaming”, “quantity of form”, “size of foam”, and “speed at which foam disappears” in the results of experiments described above indicate the “state of the frying oil Y” in use for deep-fry cooking.

Among the results of experiments described above, the evaluation items in which the graphs showing the two evaluation results have the waveforms similar to each other, and also the correlation between the state and the acid values is strong are included in the followings.
“Intensity of foaming” in the third period
“Quantity of form” in the third period
“Size of foam” in the third period
“Speed at which foam disappears” in the third period
“Size of foam” in the fourth period

Based on the above, the state indicated by, for example, the “intensity of foaming”, includes the evaluation item strongly correlated with the rate of deterioration of an acid value and the like. Note that the correlation was determined to be strong when “R” is equal to or more than “0.7”.

Furthermore, the state indicated by, for example, the “intensity of foaming” includes the item in which the waveforms of the graphs representing the evaluation results become substantially the same to each other even if the evaluators are different persons, in other words, the item which is evaluated in the same manner by each evaluator. Therefore, using the item as described above enables reduction in the influence by subjectivity of an evaluator, and thus stabile quantification of the state.

Still further, even among the same items, using the state identified in the third period in which the correlation is stronger than others enables AI prediction of the rate of deterioration with higher accuracy.

As a result of experiments described above, the correlation between the state and the rate of deterioration has been found. In particular, the rate of formation of bubbles formed per predetermined time in the edible oil, the number of bubbles, the size of a bubble, and the speed at which bubbles disappear, which are evaluated by the “intensity of foaming”, “quantity of form”, “size of foam”, and “speed at which the bubbles disappear”, respectively, are often highly correlated with the rate of deterioration.

This enables AI, upon analysis of images or input of a parameter indicating the state, to recognize the state. Then, the correlation between the state and the rate of deterioration allows AI to predict the rate of deterioration depending on the state. Therefore, the learning device 5 can generate AI capable of predicting the rate of deterioration by making the AI learn images, states, rates of deterioration, and correlations between states and rates of deterioration.

### Example of Functional Configuration

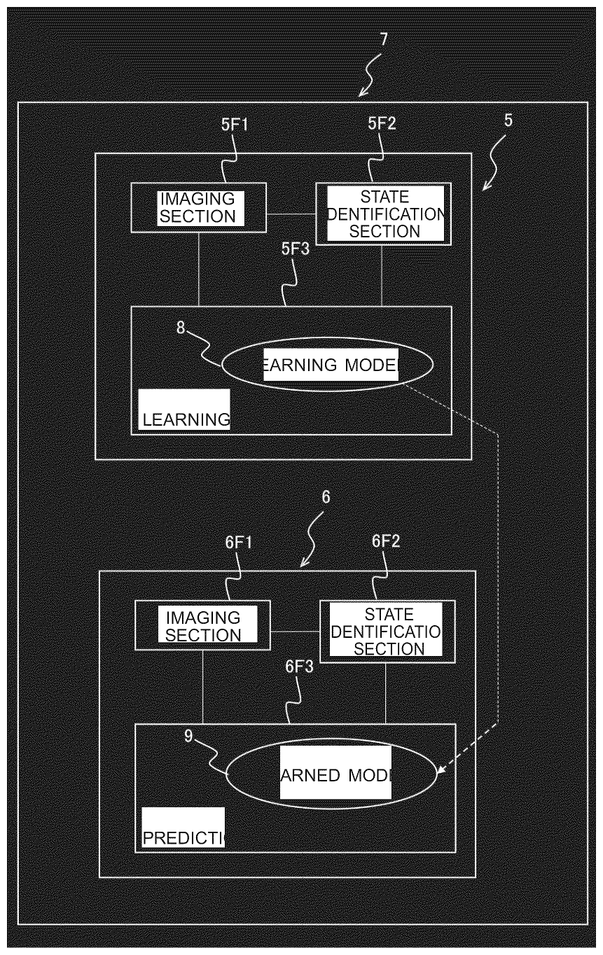

FIG. 38 illustrates an example of a functional configuration of the learning device. For example, the learning device 5 includes an imaging section 5F1, a state identification section 5F2, and a learning section 5F3.

The imaging section 5F1 carries out an imaging procedure of acquiring an image in which the edible oil is captured. For example, the imaging section 5F1 is implemented by the video camera 42, the I/F 500E, or the like.

The state identification section 5F2 analyzes the image and carries out a state identification process of identifying the state of the edible oil. For example, the state identification section 5F2 is implemented by the CPU 500A or the like.

The learning section 5F3 carries out a learning process of causing the learning model 8 to learn the correlation between the state and the rate of deterioration. For example, the learning section 5F3 is implemented by the CPU 500A or the like.

With the configuration described above, the learning device 5 causes the learning model 8 to learn so as to generate the learned model 9.

The learned model 9 is distributed to, for example, a prediction device 6 via a network or the like.

The prediction device 6 includes, for example, an imaging section 6F1, a state identification section 6F2, and a prediction section 6F3.

For example, the imaging section 6F1 has the same configuration as that of the imaging section 5F1, etc.

For example, the state identification section 6F2 has the same configuration as that of the state identification section 5F2, etc.

The prediction section 6F3 predicts the rate of deterioration using the learned model 9. For example, the prediction section 6F3 is implemented by the CPU 500A or the like.

A learning system 7 is a learning system including the learning device 5 and the prediction device 6. That is, the learning system 7 is a system that carries out both learning and prediction. For example, the learning system 7 may further cause the learned model 9 to learn after distribution of the learned model 9. Such further learning enables generation of AI which is more applicable to the individual environment of each prediction device 6.

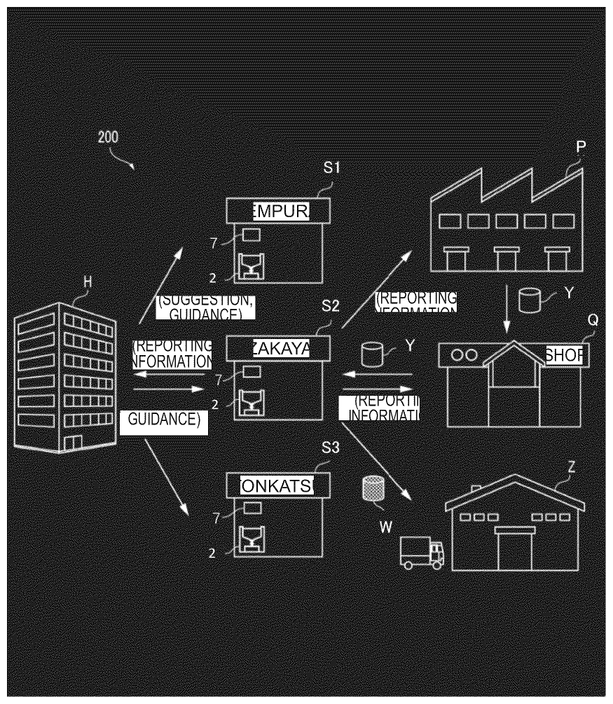

FIG. 39 illustrates an example of use of the learning system 7.

For example, a shop S2 (in this example, izakaya) notifies a headquarters H with reporting information. In this case, the headquarters H analyzes the number of times or frequency of receiving the reporting information. The headquarters H analyses in the same manner for a shop S1 (in this example, tempura restaurant) or a shop S3 (in this example, tonkatsu restaurant).

Based on the result of analysis thus obtained, the headquarters H provides suggestions or guidance as to whether the edible oil is appropriately used, appropriately changed, and efficiently used.

The headquarters H may manage the factories in which fryers are installed. The headquarters H may also manage each fryer 2 installed in equipment of the stores, shops, or factories.

A manufacturer P of edible oil and a seller Q of edible oil are also notified of the reporting information. Upon receiving the reporting information, the manufacturer P forms a manufacturing plan or a sales plan for edible oil.

Upon receiving the reporting information, the seller Q orders and purchases edible oil from the manufacturer P. Then, the seller Q distributes the edible oil to the shop S1, shop S2, and shop S3.

Still further, a disposal company Z (note that the disposal company Z and the manufacturer P may be the same) of edible oil is also notified of the reporting information. Upon receiving the reporting information, the disposal company Z arranges collection of waste oil W. Specifically, when receiving the reporting information for a predetermined number of times, an operator from the disposal company Z visits the shop S2 to collect the waste oil W from the oil vat 21 of the fryer 2.

Still further, a cleaning company (not illustrated) may also be notified of the reporting information. Upon receiving the reporting information, a cleaning operator visits the shop S2 to clean the inside of the oil vat 21 of the fryer 2 and therearound.

Thus, using the reporting information enables quick operations including supply of edible oil, disposal thereof, and cleaning in the shops S1 to S3. Furthermore, automating the change of edible oil in the shops and stores enables reduction in the burden on a user (employee in the shops and stores). Specifically, outputting the reporting information indicating that the rate of deterioration of the edible oil exceeds a threshold value causes change of the edible oil.

### Example of Network Architecture

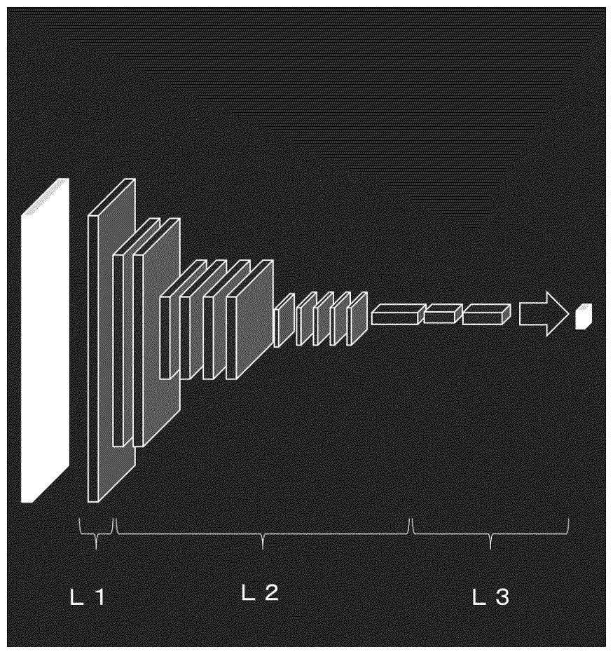

FIG. 40 illustrates an example of a network architecture. For example, AI by the learning model 8 and the learned model 9 processes image data and the like with the network architecture as follows.

For example, AI may have a network architecture including an input layer L1, a hidden layer L2, and an output layer L3.

Specifically, AI has a network architecture including a Convolution Neural Network (CNN) or the like as illustrated in FIG. 40.

The input layer L1 is a layer for inputting an image.

The hidden layer L2 is a layer for processing such as convolution, pooling, normalization, or a combination thereof with respect to the image input in the input layer L1.

The output layer L3 is a layer for outputting a result obtained by the processing by the hidden layer L2. For example, the output layer L3 is configured with a fully-connected layer or the like.

Convolution is the process for generating a feature map, based on, for example, a filter, a mask, a kernel (hereinafter, simply referred to as “filter”), or the like, by filtering on an image or filtering a feature map generated by a predetermined process on the image.

Specifically, the filter is data used for multiplying a pixel value of an image or feature map by a filter coefficient (may be referred to as “weight” or “parameter”). The filter coefficient is a value defined by learning, setting, or the like.

The convolution process is the process of multiplying a pixel value of each pixel forming the image or feature map by a filter coefficient to generate a feature map having a calculation result as its component.

As a result of the convolution process described above, features of the image or those of the feature map can be extracted. The features are, for example, edge components or a result of statistical process of a periphery of a target pixel.

Furthermore, as a result of the convolution process, even from an image or feature map in which a subject indicated thereby is vertically shifted, horizontally shifted, obliquely shifted, rotated, or have an attitude of a combination thereof, the similar features can be extracted.

Pooling is the process of extracting features to generate a feature map by performing the process such as calculation of an average, extraction of a minimum value, or extraction of a maximum value on a target area. That is, pooling is max pooling, avg pooling, or the like.

Note that convolution and pooling may include preprocessing such as Zero Padding.

By performing the processes described above such as convolution, pooling, or a combination thereof, so-called effects of reduction in data amount, compositionality, translation invariance, or the like can be obtained.

Normalization is the process of, for example, equalizing variances and averages. Note that normalization may be performed locally. Normalization causes the data to have values within a predetermined range. This makes the data to be easily handled in subsequent processing.

Fully connected is the process of dropping data, such as a feature map or other data, into the output.

For example, data is output in the binary format, such as “YES” or “NO”. In this type of output format, fully-connected is the process of joining the nodes based on the features extracted in the hidden layer L2 so that either of the two types is the conclusion.

On the other hand, in the case of three or more types of outputs, fully connected is the process of performing a so-called soft max function or the like. As described above, the fully connected process allows classification (including outputs indicating probabilities) to be performed by the maximum likelihood estimation.

### Results of Experiments Using Edible Oil Longer

As described above, using the learning model having learned the correlation between the state and the rate of deterioration allows the prediction device 6 to predict the rate of deterioration based on the state.

The following is the results of experiment of investigating how edible oil deteriorates if further using the edible oil from the point of time of the prediction to confirm whether the prediction by the prediction device 6 can be established. In the experiments, the following fried foods were used.

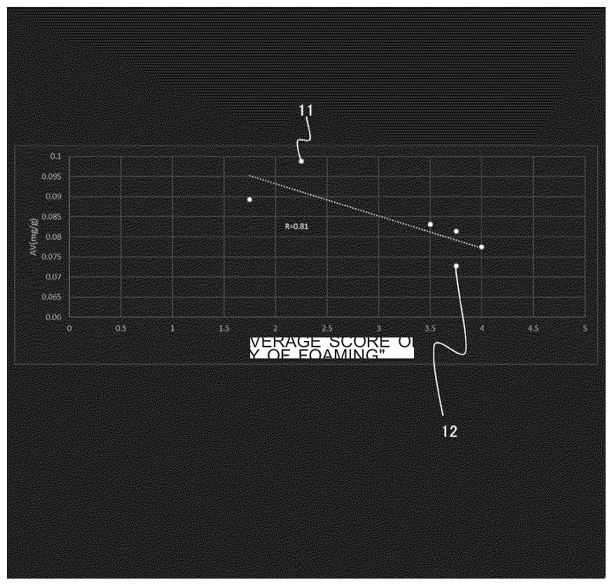

FIG. 41 illustrates the fried foods which are subjects of experiments. FIG. 41 is similar to FIG. 23. In the following, among the samples showing the results of experiments illustrated in FIG. 41, the results of experiment using a first sample 11 and a second sample 12 as subjects of experiments will be described.

As illustrated in the results of experiments in FIG. 23, the first sample 11 is a fried food having a lower evaluation score of “intensity of foaming” than that of the second sample 12. That is, foaming in the edible oil is likely to be relatively mild during deep-fry cooking of the first sample 11.

Based on the correlation found from the results of experiments illustrated in FIG. 23, the state such as “intensity of foaming” has a correlation that the lower the evaluation value is, the higher the acid value is, and thus the rate of deterioration easily increases.

Accordingly, the prediction device 6 predicts that the rate of deterioration is likely to increase higher in the case of the first sample 11 than the case of the second sample 12 based on the learning of the correlation. For verifying whether this prediction result is correct, the following experiment was carried out.

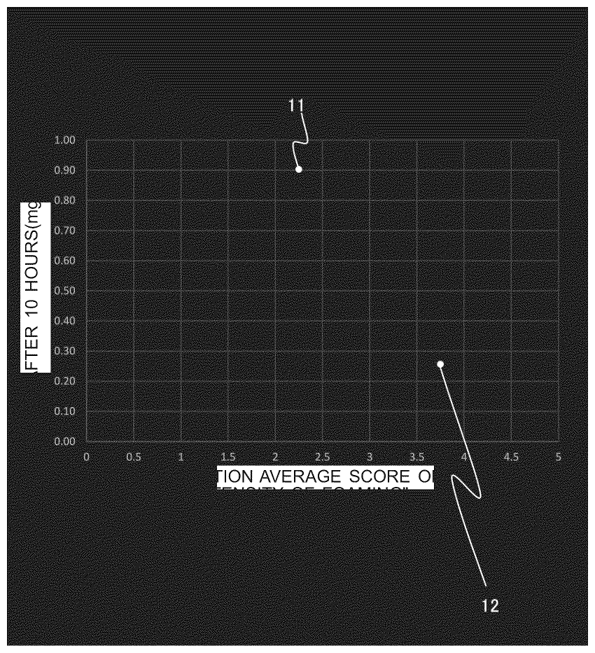

FIG. 42 illustrates the results of experiments of measuring the acid values after 10 hours. The results of experiments illustrated in FIG. 42 show the ones obtained by continuously deep-frying the first sample 11 and second sample 12 for 10 hours. In the experiments, a total of 160 pieces of the first sample 11 and second sample 12 were cooked.

The acid value when the first sample 11 was continuously cooked for 10 hours was “0.90”.

The acid value when the second sample 12 was continuously cooked for 10 hours was “0.26”.

Specifically, the rate of deterioration after deep-frying for 10 hours changed as follows.

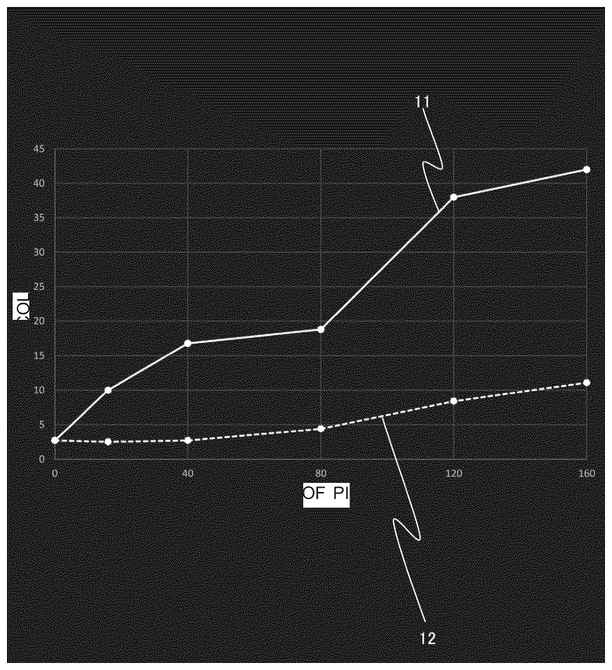

FIG. 43 illustrates the results of experiments using color. In FIG. 43, the horizontal axis represents the number of fried foods that have been deep-fried while the vertical axis represents the results of measurement obtained by measurement of the color. The color is a value calculated by “the yellow component value plus 10×the red component value”. That is, the higher the value of the color, the higher the rate of deterioration becomes, which shows that the edible oil deteriorates.

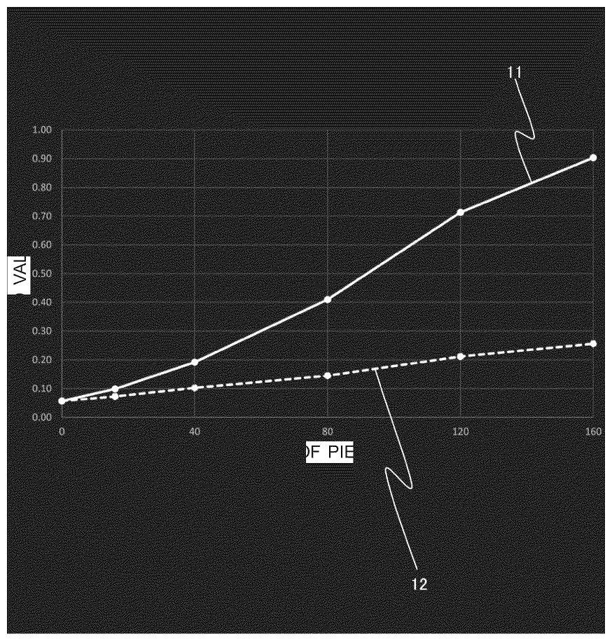

FIG. 44 illustrates the results of experiments using acid values. The horizontal axis is the same as that of FIG. 43. On the other hand, the vertical axis represents the results of measurement obtained by measurement of the acid values. That is, the higher the acid value is, the higher the rate of deterioration becomes, which shows that the edible oil deteriorates.

As illustrated in FIG. 43 and FIG. 44, in both the experiments, the rate of deterioration in the case of the first sample 11 was higher than the case of the second sample 12.

Thus, having learned the correlation and recognizing the state of the relatively new edible oil allows the prediction device 6 to predict how the rate of deterioration increases after 10 hours based on the correlation.

### Modifications

In the prediction, for example, the shelf life, weight of a fried food, temperature, humidity, size, arrangement of fried foods during deep-fry cooking, thickness, ratio of batter-coating, raw materials used in foods, moisture content, or a combination thereof may be considered.

The result of prediction may be output in the format of indicating the tendency of deterioration or in the format in which when to change the edible oil, such as whether it is time to change the edible oil, is predicted.

For example, the result of prediction is displayed on the monitor 41, like “current rate of deterioration is 00%”. That is, the monitor 41 shows the current level expressed by a percentage where the percentage of the time to change the oil is “100%”. On the other hand, when the result of prediction of the rate of deterioration indicates that the time to change has been reached, the monitor 41 may display, for example, the result of prediction by displaying a message like “please change frying oil”.

When the type of fried food X to be deep-fried next and the number of pieces for each type are input or estimated, the monitor 41 displays, for example, “○ more pieces to be deep-dried are left”, “you can deep-fry ○ pieces of ○○or • pieces of •• for next time”, “add new oil now, and you can use this oil for ○ more days”, and the like. That is, the monitor 41 may display the prediction result in the format of informing the content, for example, the type and number of fried foods, capable of being cooked until the time to change the oil has reached.

Analyzing an image may enable calculation of the “number of bubbles”, “size of a bubble”, “ratio of the area where bubbles each having the predetermined size are formed relative to the total area”, “time from formation of the specific bubbles to disappearance thereof (speed of disappearance) “, or a combination thereof.

Furthermore, using these results of calculation, images, or a combination thereof may enable identification of the “acid value”, “color tone”, “rate of increase in viscosity “, “degree of flow of bubbles”, “visibility of the outline in an image of a fried food”, type of frying oil, type of a fried food, quantity of fried foods, a combination thereof, or the like.

### New Oil and Initial Foam

Preferably, the state is identified based on the foam (hereinafter, referred to as “initial foam”) formed in deep-fry cooking using new edible oil (hereinafter, referred to as “new oil”).

The new oil referred herein is the edible oil having a low acid value. Specifically, the new oil is the edible oil having an acid value of less than 0.2. That is, the new oil is completely unused edible oil or edible oil less frequently used.

In many cases, the acid value is not more than 0.2 when heating time thereof is several tens of hours or less. Accordingly, in the present specification, whether the edible oil corresponds to “new oil” is mainly identified based on the acid value.

The initial foam referred herein is the foam formed in the new oil. For example, the initial foam is often the foam formed when the frequency of use of the new oil in deep-fry cooking is less than 10 times.

The acid value serving as a reference for determining whether the oil is the new oil may be other than “0.2”, depending on the initial value of the acid value or the type of edible oil. Furthermore, the predetermined frequency of deep-fry cooking serving as a reference for determining whether the foam is the initial foam may be other than “10 times”, depending on the type of fried food or the like.

Still further, whether the oil is new oil may be evaluated based on a reference other than the acid value. For example, whether the oil is new oil may be evaluated based on the frequency of use, use time, and the like. It may be evaluated based on a plurality of items.

As described above, it is preferable that the state of the edible oil is identified based on the initial foam. Recognizing the state of the edible oil using the initial foam as described above enables AI to accurately predict the rate of deterioration of the edible oil during use.

### Other Embodiments

Other embodiments in which the embodiments described above are combined each other may be adopted.

In an exemplary embodiment, the process of reducing over-learning (also referred to as “over-fitting”) such as dropout may be performed. In addition, the processes such as dimension reduction and normalization may be performed.

The network architectures of the learning model and learned model are not limited to CNN. For example, the network architecture may have a configuration such as RNN (Recurrent Neural Network) or LSTM (Long Short-Term Memory).

Furthermore, the learning model and the learned model may have a configuration including hyper parameters. That is, the learning model and the learned model may allow a user to set a part of the settings.

Still further, the learning model and the learned model may use other types of machine learning. For example, the learning model and the learned model may perform the pre-processing such as normalization by using an unsupervised model.

The present invention may be implemented by the learning method exemplified above or a program for executing the processing equivalent to the processing described above (including firmware and one equivalent to the program, and hereinafter, simply referred to as “program”).

That is, the present invention may be realized by a program or the like described in a programming language or the like so that a predetermined result is obtained by executing a command to a computer. The program may be configured to execute a part of the process by hardware such as an integrated circuit (IC) or an computing device such as a GPU.

The program causes a computer to execute the processing described above by making the computing device, control device, and storage device equipped in the computer cooperate. That is, the program is loaded onto the main storage device or the like and then issues a command to cause the computing device to execute the calculation, thereby causing the computer to operate.

Furthermore, the program may be provided via a computer-readable recording medium or a telecommunication line such as a network.

The present invention may be realized by a system including a plurality of devices. That is, the information processing system by a plurality of computers may execute the processing described above by a redundant, parallel, or distributed system, or a combination thereof. Accordingly, the present invention may be realized by a device having a configuration other than the hardware configuration described above and a system other than the one described above.

In the above, the present invention has been described with reference to the embodiments of the present invention. The present invention is not limited to the embodiments described above, and various modifications may be made therein. For example, each of the embodiments is described in detail herein for the purpose of clarity and a concise description, and the present invention is not necessarily limited to those including all the features described above. Furthermore, some of the features according to a predetermined embodiment can be replaced with other features according to the separate embodiments, and other features can be added to the configuration of a predetermined embodiment. Still further, some of the features can include other features of the separate embodiments, be deleted, and/or replaced.

### REFERENCE SIGNS LIST

1: cooking area
2: fryer
3: fry basket
5: learning device
5F1: imaging section
5F2: state identification section
5F3: learning section
6: prediction device
6F1: imaging section
6F2: state identification section
6F3: prediction section
7: learning system
8: learning model
9: learned model
41: monitor
42: video camera
L1: input layer
L2: hidden layer
L3: output layer
α: bubble
β: bubble

1. A learning device comprising:
an imaging section configured to acquire an image in which edible oil is captured;
a state identification section configured to analyze the image to identify a state of the edible oil; and
a learning section configured to cause a learning model to learn a correlation between the state and a rate of deterioration indicating an increased level of deterioration of the edible oil.

2. The learning device according to claim 1, wherein
the state is a rate of formation of bubbles formed per predetermined time in the edible oil, the number of the bubbles, size of each of the bubbles, speed at which the bubbles disappear, or a combination thereof.

3. The learning device according to claim 1, wherein
the rate of deterioration is a rate of increase in viscosity of the edible oil, an acid value of the edible oil, color of the edible oil, an Anisidine value of the edible oil, the quantity of polar compounds of the edible oil, a Carbonyl value of the edible oil, a smoke point of the edible oil, Tocopherol content of the edible oil, an iodine value of the edible oil, a refractive index of the edible oil, the quantity of volatile compounds of the edible oil, a composition of volatile compounds of the edible oil, flavor of the edible oil, the quantity of volatile compounds of a deep-fried food obtained by deep-fry cooking with the edible oil, a composition of volatile compounds of the deep-fried food, flavor of the deep-fried food, or a combination thereof.

4. The learning device according to claim 1, wherein
the state is identified based on initial foam formed in edible oil having an acid value of less than 0.2.

5. A prediction device comprising a prediction section configured to predict the rate of deterioration using a learned model that is the learning model made to learn by the learning device according to claim 1.

6. A method of learning carried out by a learning device, comprising:
an imaging procedure in which the learning device acquires an image in which edible oil is captured;
a state identification procedure in which the learning device analyzes the image to identify a state of the edible oil; and
a learning procedure in which the learning device causes a learning model to learn a correlation between the state and a rate of deterioration indicating an increased level of deterioration of the edible oil.

7. A computer readable media for causing a computer to execute the learning method according to claim 6.

8. A learning system comprising:
the learning device according to claim 1; and
the prediction device according to claim 5.
